SEQUENCE LISTING (24 sequences)

SEQ ID NO: 1 - DNA, 20 bp, Mus musculus
cactgagcatctccctcaca

SEQ ID NO: 2 - DNA, 20 bp, Mus musculus
tgggtgcagcgaactttatt

SEQ ID NO: 3 - DNA, 20 bp, Mus musculus
agcctgaagactgtgatggg

SEQ ID NO: 4 - DNA, 19 bp, Mus musculus
aaagttccaccgttctcgg

SEQ ID NO: 5 - DNA, 22 bp, Mus musculus
tgtttgtaaagctgccgtctga

SEQ ID NO: 6 - DNA, 19 bp, Mus musculus
cctgcctgttcccggtatc

SEQ ID NO: 7 - DNA, 20 bp, Mus musculus
ctggatgagggctcagatgt

SEQ ID NO: 8 - DNA, 20 bp, Mus musculus
ggttagctcctgcctgctta

SEQ ID NO: 9 - DNA, 20 bp, Mus musculus
gagttcgattagccgagtgc

SEQ ID NO: 10 - DNA, 20 bp, Mus musculus
ggttagctcctgcctgctta

SEQ ID NO: 11 - DNA, 20 bp, Mus musculus
agacgcctgaagaaggtcaa

SEQ ID NO: 12 - DNA, 20 bp, Mus musculus
agctggacacggagctttta

SEQ ID NO: 13 - DNA, 20 bp, Mus musculus
aaccatatccgccacaagat

SEQ ID NO: 14 - DNA, 20 bp, Mus musculus
ctagatccgcctcctcctct

SEQ ID NO: 15 - DNA, 20 bp, Mus musculus
tacccaaggtggagatcctg

SEQ ID NO: 16 - DNA, 20 bp, Mus musculus
gtggagatgcgctccactat

SEQ ID NO: 17 - DNA, 20 bp, Mus musculus
gcgagaagaaagccaaacac

SEQ ID NO: 18 - DNA, 20 bp, Mus musculus
gggtgtagatgtccgggtag

SEQ ID NO: 19 - DNA, 20 bp, Mus musculus
ccaccaaccctaaccagaga

SEQ ID NO: 20 - DNA, 20 bp, Mus musculus
ctgttctttcgggaccagac

SEQ ID NO: 21 - DNA, 20 bp, Mus musculus
cccatggttgcgtctctaag

SEQ ID NO: 22 - DNA, 20 bp, Mus musculus
ggatgcggctgatagaactc

SEQ ID NO: 23 - DNA, 19 bp, Mus musculus
gccttctacgcacctggac

SEQ ID NO: 24 - DNA, 20 bp, Mus musculus
actcttccctggcctggact

(19) United States
(12) Patent Application Publication
(10) Pub. No.: US 2012/0276064 A1
(43) Pub. Date: Nov. 1, 2012

(54) METHODS AND COMPOSITIONS FOR REJUVENATION AND EXPANSION OF STEM CELLS

(76) Inventors: Helen M. Blau, Menlo Park, CA (US); Benjamin D. Cosgrove, Stanford, CA (US); Penney M. Gilbert, Menlo Park, CA (US)

(21) Appl. No.: 13/440,534

(22) Filed: Apr. 5, 2012

Related U.S. Application Data
(60) Provisional application No. 61/472,121, filed on Apr. 5, 2011.

(51) Int. Cl. A61K 31/7088; A61K 35/30; A61K 35/34; A61K 35/407; A61K 35/37; A61K 35/28; A61K 35/14; A61K 35/44; A61K 35/32; A61K 35/39; A61K 35/12; A61K 35/36

(52) U.S. Cl. 424/93.7; 514/44 R

(57) ABSTRACT

The regenerative potential of stem cells is enhanced in vitro and in vivo by inhibition of the p38 MAPK signaling pathway.

### BACKGROUND OF THE INVENTION

Stem cells have a capacity both for self-renewal and the generation of differentiated cell types, which provides the possibility for therapeutic regeneration of cells and tissues in the body. In addition to studying the important normal function of stem cells in the regeneration of tissues, researchers have further sought to exploit the potential of in situ and/or exogenous stem cells for the treatment of a variety of disorders. While early, embryonic stem cells have generated considerable interest, the stem cells resident in adult tissues also provide an important source of regenerative capacity.

These somatic, or adult, stem cells are undifferentiated cells that reside in differentiated tissues, and have the properties of both self-renewal and generation of differentiated cell types. The differentiated cell types may include all or some of the specialized cells in the tissue. For example, hematopoietic stem cells give rise to all hematopoietic lineages, but do not seem to give rise to stromal and other cells found in the bone marrow. Sources of somatic stem cells include bone marrow, blood, the cornea and the retina of the eye, brain, skeletal muscle, cartilage, bones, dental pulp, liver, skin, the lining of the gastrointestinal tract, and pancreas, and the like. Adult stem cells are usually quite sparse. Often they are difficult to identify, isolate, and purify. Often, somatic stem cells are quiescent until stimulated by the appropriate growth signals.

Somatic stem cells in vivo reside in so-called “niches” or protective microenvironments that are composed of complex mixtures of signaling proteins. A specific microenvironment, or niche, has been shown to play a critical role in the maintenance of stem cell functions, for example see Spradling et al., Nature 414: 98 (2001); Fuchs et al., Cell 116: 769 (2004); Moore & Lemischka, Science 311: 1880 (2006); Scadden, Nature 441: 1075 (2006). Many essential signals may be membrane-bound and thus conformationally controlled and immobilized on supportive cells in close physical contact with adult stem cells. These signals direct stem cell behavior by different means, protecting them from differentiation, influencing the cell cycle (e.g., maintaining quiescence) and self-renewal divisions. In the absence of cross-talk with their respective natural niche, as is the case with in vitro culture, adult stem cells rapidly differentiate and lose their multipotentiality.

Muscle tissue in adult vertebrates regenerates from stem cells known as satellite cells. Satellite cells are distributed throughout muscle tissue and are mitotically quiescent in the absence of injury or disease, residing in an instructive, anatomically defined niche. The satellite cell niche constitutes a distinct membrane-enclosed compartment within the muscle fiber, containing a diversity of biochemical and biophysical signals that influence satellite cell function. In addition to satellite cells, cell types that contribute to muscle regeneration include mesangioblasts, bone marrow derived cells, muscle interstitial cells, mesenchymal stem cells, etc. See D. D. Cornelison et al. (2001) Dev Biol 239, 79; S. Fukada et al. (2004) Exp Cell Res 296, 245; D. Montarras et al. (2005) Science 309, 2064; S. Kuang et al. (2007) Cell 129, 999; M. Cerletti et al. (2008) Cell 134, 37; C. A. Collins et al. (2005) Cell 122, 289; A. Sacco et al. (2008) Nature 456, 502; R. I. Sherwood et al. (2004) Cell 119, 543; Sampaolesi et al. (2003) Science 301(5632):487-92; and Galvez et al. (2006) J Cell Biol. 174(2):231-43.

An important aspect of stem cell biology is the effect of aging. Many tissues of the major organ systems are composed of long-lived cells that are normally replaced infrequently, if at all. Therefore, such cells persist for significant portions of a mammal's lifespan. The functional activity of these cells changes over time in a process that is generally referred to as aging and, as most age-related changes in cells result in reduced functional viability, this can be harmful.

Many of the pathophysiological conditions afflicting the elderly, such as anemia, sarcopenia (loss of muscle mass), and osteoporosis, suggest an imbalance between cell loss and renewal. The fact that homeostatic maintenance and regenerative potential of tissues wane with age has implicated stem cell decline as a central player in the aging process. The degree to which aging is attributable to stem cell dysfunction or instead reflects a more systemic degeneration of tissues and organs may vary between different tissues and their resident stem cells. Nonetheless, mounting evidence points to stem cells as an important contributing factor to at least some of the pathophysiological attributes of aging in a number of different tissues. (see Rossi et al. (2008) Cell 132(4):681-696).

For example, advancing age is accompanied by a number of pathophysiological changes in the hematopoietic system, suggesting that the proliferative and regenerative capacity of human HSCs diminishes with age, and that diminished stem cell activity is largely cell intrinsic.

Graying of hair, one of the most recognizable aspects of human aging, appears to result from an age-dependent loss of melanocyte stem cells (MSCs) from the subcutaneous hair follicle bulge region (Nishimura et al., 2005) and may be exacerbated by telomere dysfunction as suggested by the premature graying of telomerase-deficient mice (Rudolph et al., 1999).

The decline in both sensory and cognitive functions in the elderly implicates age-associated neural stem cell decline as an important contributory factor. In support of this, NSC numbers and proliferative potential are reduced in the SVZ with age, correlating with the diminished neurogenesis observed in the olfactory bulb of old mice.

The age-related decrease in central nervous system remyelination efficiency has been attributed to an impairment of oligodendrocyte progenitor cell recruitment and differentiation, Sim et al. (2002) J Neurosci. 22(7):2451-9. The progenitor cell response during remyelination of focal, toxin-induced CNS demyelination in young and old rats was compared and found to be delayed with aged animals.

The adverse effects of aging include deterioration in the ability of somatic stem cells to regenerate differentiated cells in multiple different tissues. Methods of increasing the ability of aged stem cells to regenerate tissues are of enormous clinical interest.

### SUMMARY OF THE INVENTION

Methods and compositions are provided for rejuvenation and proliferation of stem and/or progenitor cells in vivo, or in ex vivo culture. Cells, e.g. somatic stem/progenitor cells, are contacted with an inhibitor of the p38 signaling pathway in a dose effective to stimulate rejuvenation and proliferation of the targeted cells, where stem/progenitor cells obtained from aged, diseased, and/or injured individuals are of particular interest. Rejuvenation of a stem/progenitor cell may involve re-entry into cell cycle; proliferation; and subsequent differentiation of progeny cells into appropriate specialized cells and tissues, particularly an increase in the ability of stem cells to repair the cognate tissue. In some embodiments a stem cell from other than an aged individual is expanded in the presence of a p38MAPK signaling pathway inhibitor, e.g. by expansion in an ex vivo setting as described herein.

In one embodiment of the invention, a subject who is in need of tissue rejuvenation, e.g. due to an injury, disease, or aging, is contacted with an inhibitor of the p38 mitogen-associated protein kinase (MAPK) signaling pathway in a dose effective to stimulate rejuvenation and proliferation of stem cells resident in the targeted tissue. Included as inhibitors of interest are molecules that selectively inhibit p38 MAPK, a component of the MAPK signaling pathway, or a component upstream or downstream of the MAPK signaling pathway. Inhibitors of interest may be small molecules, polynucleotides, antibodies, antagonist proteins, and the like. Inhibitors of interest include, without limitation, small molecule inhibitors of the α and β isoforms of p38 MAP kinase, e.g. SB202190, SB203580, etc. Delivery of the inhibitor may be systemic or localized, usually localized.

In some embodiments, the tissue is muscle tissue. Resident in muscle tissue are satellite cells, which are stem cells that ultimately give rise to muscle fibers. Satellite cells in aged muscle are rejuvenated and capable of regeneration of tissue when the p38 MAPK signaling pathway is inhibited.

In another embodiment, the tissue is neural tissue, including hippocampal tissue, where neural stem cells are resident, and are activated by the methods of the invention. In another embodiment, the tissue is hematopoietic, and hematopoietic and/or mesenchymal stem cells are activated. In another embodiment, the tissue is liver tissue, including hepatocytes and liver stem cells, and the like.

A tissue regenerative agent, which comprises at least one inhibitor of the p38 MAPK signaling pathway may be provided in a pharmaceutical formulation suitable for administration to a patient. Formulations of interest include those that provide for substantial retention of the agent in the tissue of interest or formulations that can be localized into tissues of interest by localized delivery, e.g. antibody conjugates that directly and specifically target cells of interest; or localized activation.

In some aspects of the invention, methods are provided for the expansion of stem/progenitor cells, e.g. somatic stem cells including somatic stem cells obtained from an aged individual, in culture in the presence of a p38 signaling pathway inhibitor in a dose effective to stimulate rejuvenation and proliferation of the targeted stem cells. Optionally, the stem cells are seeded in vitro on the surface of a pliable substrate having an elasticity of a physiological substrate, for example an elasticity that is matched to the elasticity of the tissue from which the cells are derived For example, an elasticity of from about 1 to about 100 kPa may be utilized for the expansion of muscle stem cells. Substrates of interest include, synthetic or artificial hydrogels, including without limitation matrigel, collagen, fibronectin, and the like as a substrate surface. In some embodiments the hydrogel further comprises at least one polypeptide or chemical component, e.g. a structural or soluble protein. The expanded stem cell population may be utilized for in vivo purposes, including transplantation for cell based therapies; for various screening purposes in vitro, for modeling and profiling healthy and disease phenotypes in culture, and the like.

The cells may be maintained in culture for a period of time sufficient to increase the number of cells having a stem cell phenotype, for example increasing the number of stem cells by at least 1.5 fold, at least two-fold, at least three-fold, at least five-fold, at least 10-fold, or more. Cultures may be maintained for a period of time sufficient to observe the parameters of interest, for example at least at least about an hour, about 2 hours, about 6 hours, about 1 day, about 1 week, about 2 weeks, about 3 weeks, or more. Stem cell phenotypes of interest include, without limitation, the ability to participate in the development of the cognate tissue in vivo; display of cell surface markers shown to be associated with the cognate stem cell; ability to give rise in vitro to colonies of the differentiated cell types associated with the stem cell; and the like.

In certain embodiments of the invention, the stem cell is a somatic cell. In some cases, the somatic cell is an aged muscle stem cell, which cells are shown herein to require for proliferation in vitro a pliable substrate. The substrate for muscle stem cell expansion may further comprise at least one protein component of the muscle tissue, or niche, e.g. laminin, fibronectin, etc. It is shown herein that muscle stem cells cultured by the methods of the invention are able to contribute to in vivo tissue regeneration. The expanded population of muscle stem cells is useful in transplantation, particularly for the regeneration of skeletal muscle, e.g. in the treatment of muscle disorders such as heritable or acquired muscular dystrophies, myopathies, chanelopathies, sarcopenia; following traumatic damage; and the like.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Diminished regenerative potential of transplanted old MuSCs reveals an inherent stem cell defect. (A) Flow cytometric purification of MuSCs from young and old mice. (B-C) Nested RT-PCR analysis of FACS-sorted single young (n=45) and old (n=31) MuSCs. Ten representative gels shown in (B). No comparisons significant by Fishers exact test in (C). (D-H) Bioluminescence imaging (BLI) and GFP immunohistological analysis (in transverse sections) one month after transplantation of young or old MuSCs (purified from GFP/Luciferase mice) into tibialis anterior muscles of recipient mice. (D) BLI signals from 100-cell (n=15) and 10-cell (n=34) transplants from three independent experiments. BLI engraftment threshold defined by corresponding histological detection of donor-derived (GFP⁺) myofibers (dashed line; see also FIG. S4A). (E) Representative BLI images. (F) Representative immunohistological images. Scale bar, 100 μm. (G) Percentage of transplants above engraftment threshold. P<0.05 by Fishers exact test. (H) Limiting dilution analysis relating MuSCs transplanted with percent engraftment.

FIG. 2. p38 inhibition promotes proliferation and stem-cell gene expression of young and old MuSCs cultured on soft hydrogels. (A-C) MuSCs purified from young and old GFP/Luciferase mice were cultured on rigid (˜10⁶ kPa) or soft (12 kPa) hydrogels for one week and then 100 cells were transplanted into recipient mice. n=18 and 31 for young MuSCs and n=10 and 31 for old MuSCs on rigid and soft hydrogels, respectively, from three independent experiments. (A) Bioluminescence imaging (BLI) signals at one-month post-transplant. Dashed line is engraftment threshold. (B) Representative BLI images. (C) Percentage of transplants above engraftment threshold. P<0.05 by Fisher's exact test. (D) Clonal proliferation of young and old MuSCs in soft hydrogel microwell cultures over four days. Scale bar, 100 μm. (E) Percentage of single cells (n>70 across two independent experiments) that divided. P<0.05 by Fisher's exact test. (F-I) Young and old MuSCs cultured on soft hydrogels without (−) or with 10 μM SB202190 (SB) for one week. Mean±s.e.m. from four independent experiments. P<0.05 by t test. (F) Increase in total cell numbers (relative to seeding). (G-I) RT-PCR gene expression analysis of p21 (G), Myogenin (H), and Pax7 (I). Transcript levels normalized to Gapdh and plotted relative to purified young MuSCs (see FIG. S8A-C). (J) Effects of p38 inhibition on MuSC cell cycle and self-renewal in hydrogel culture. ‘X’ indicates a regulatory connection attenuated by SB.

FIG. 3. p38 inhibition promotes rejuvenation and expansion of old MuSCs cultured on soft hydrogels. (A-C) Young and old GFP/Luciferase MuSCs were cultured on soft (12 kPa) hydrogels without (−) or with 10 μM SB202190 (SB) for one week then 100 cells were transplanted into recipient mice. n=44 for young and n=32 for old MuSCs from three independent experiments. (A, F) Bioluminescence imaging (BLI) signals at one-month transplant. Dashed line is engraftment threshold. (B) Representative BLI images from (A). See FIG. S12A for images representative of (F). (C, G) Percentage of transplants above engraftment threshold. P-values (*P<0.01) from Fishers exact test. (D) Limiting dilution model predicts transplant engraftment frequency from varying numbers of fresh MuSCs or the progeny of equivalent numbers of cultured MuSCs (see also FIG. S11). (E-G) Expansion assay comparing the functional stem cell content in 10 young or old freshly isolated MuSCs to the progeny of 10 old MuSCs (˜300 cells) after one week on soft hydrogels+SB (n=24). (H) Immunohistochemistry of transverse sections from notexin-injured muscle one-month after old ^{Myf}5^{nLacZl+} (15, 28) MuSC transplantation from one-week SB-treated soft hydrogel cultures. Arrowhead indicates Myf5-η-gal⁺ cell in satellite cell position. Scale bar, 50 μm.

FIG. 4. Transplantation of the rejuvenated and expanded progeny of old MuSCs from p38-inhibited hydrogel cultures rescues the strength of injured muscles in old mice. (A-F) Transplantation of the progeny of 100 old MuSCs cultured on soft hydrogels for one week without (Ctrl; ˜200 cells total) or with 10 μM SB202190 (SB; ˜3000 cells total) into old syngeneic muscles two days post-notexin (NTX) injury. (B) Transplant engraftment frequency at one-month post-transplant (n=8). P<0.05 by Fishers exact test. (C) Representative BLI images. (D) Transverse muscle section immunohistochemistry two months after transplantation with the culture progeny of 100 SB-treated old MuSCs with GFP lentivirus infection. Scale bar, 100 μm. (E-F) Muscle function assessed two months post-injury using in vivo measurements of specific twitch and tetanic forces. NTX-injured muscles in old (O) mice transplanted with the culture progeny of 100 control or SB-treated old MuSCs, compared to control uninjured and injured muscles in old mice and uninjured young (Y) muscles. Mean±s.e.m. from n=3-5 muscles. P<0.05 by t test.

FIG. 5. Flow cytometry isolation of MuSCs from young and old mice. (A-B) From young (A) and old (B) C57BL/6 mice, cells dissociated from digested hindlimb skeletal muscles were magnetically-depleted for blood markers CD45 and CD11b, endothelial marker CD31, and mesenchymal marker Sca1. From this depleted population, mononucleated cells (scatter plots not shown) were gated for viable cells (propidium iodide negativity; left panels) then cells negative for CD45/CD11b/CD31/Sca1 were gated (middle-left panels). Within this population, CD34+ α7-integrin+ cells were gated, representing the MuSC fraction (middle-right panels). In the first sort, the MuSC fraction routinely comprised 2-5% of all cells present in the magnetically-depleted populations. Cells were double-sorted for purity (routinely >95%; right panels). (C-D) From young (C) and old (D) GFP/Luciferase transgenic mice, MuSCs were isolated as in (A-B), except cells were also gated for GFP positivity (left panels). Cells were double-sorted for purity (routinely >90%; right panels).

FIG. 6. Nested RT-PCR analysis of muscle stem cell genes (Pax7 and Myf5) and myogenic commitment genes (Pax3 and MyoD) in single MuSCs isolated from young and old mice. Single sorted young (A; n=45 cells) and old (B; n=31 cells) MuSCs were analyzed across two independent experiments.

FIG. 7. Scheme of transplantation experiments assessing muscle stem cell regenerative function. MuSCs were prospectively isolated by flow cytometry after enzymatic digestion from young or old GFP/Luciferase transgenic mice. In some experiments isolated MuSCs were cultured for one week on hydrogel and subsequently assayed for proliferation and myogenic gene expression. In experiments designed to assess the in vivo regenerative function of young or old MuSCs, cells were transplanted into tibialis anterior muscles of hindlimb irradiated NOD/SCID recipient mice immediately after isolation (fresh) or after one week of hydrogel culture.

FIG. 8. Bioluminescence imaging at one-month post-MuSC transplantation detects stable engraftment into recipient muscle. (A) Correlation of bioluminescence imaging (BLI) signal and histological detection of donor cell-derived myofibers at one-month post-transplantation. Varying numbers of freshly isolated and cultured young and old GFP/Luciferase MuSCs were transplanted into the tibialis anterior muscle of recipient mice and assayed one month post-transplant by bioluminescence imaging (BLI) and GFP immunohistological analysis (n=11 transplants). GFP⁺ myofibers were counted on immunofluorescence images of transverse sections of recipient muscles. Transplants resulting in no detectable GFP⁺ myofibers are plotted as “non-engrafted” samples. BLI signals (note: log-scale) and number of GFP⁺ myofibers are well-correlated using a linear fit (R²=0.99). Intercept of linear fit defines the BLI signal threshold (dashed line at ˜80,000 photon/s) corresponding to the histological detection of one or more GFP⁺ myofiber (i.e., “engraftment”). Inset contains a magnified region of the plot near the engraftment threshold. (B) Long-term, stable engraftment of transplanted MuSCs. One-hundred freshly isolated MuSCs from young luciferase-expressing mice were transplanted into the tibialis anterior muscle of recipient mice and assayed for seven months post-transplant by BLI. BLI detection threshold indicated by dashed line. Mean±s.e.m from n=6 replicates (note: all transplants engrafted) are plotted.

FIG. 9. Limiting dilution analysis of MuSC transplant engraftment indicates a diminished proportion of functional stem cells in old MuSCs compared to young MuSCs. (A) Engraftment percentages of freshly isolated young and old MuSCs transplanted in varying cell numbers. (B) Data were fit to an exponential limiting dilution model, relating the engraftment percentages to the number of cells transplanted for each age of MuSC. Exponential parameter (β) and model fitness (R²) values for each experimental condition were tabulated. Limiting dilution model equations are used to derive an estimate for the effective number of functional MuSCs with engraftment potential within any number of total cells from any condition. Functional MuSCs are here defined as the freshly isolated young MuSC population.

FIG. 10. Candidate treatment analysis identifies p38 inhibitor SB202190 for its profound stimulation of proliferation of hydrogel-cultured young and old MuSCs. Young and old MuSCs were cultured on soft (12 kPa) hydrogels and treated daily with DMSO carrier control (−), 25 ng/ml Wnt7a, 25 ng/ml Jagged1-Fc (Jag1), 10 nM tautomycetin (Tauto), 10 μM SB202190 (SB202), or 1 μM SB431542 (SB431) for one week. See table S1 for details on these treatments. Increase in total cell numbers (relative to seeding) after one week of culture are plotted. Error bars represent s.e.m. from n=3 experiments. P<0.05 by t test are noted for comparisons between control and treatments for each age of MuSCs.

FIG. 11. Pretreatment with p38 inhibitor SB202190 prevents MK2 phosphorylation stimulated by anisomycin and TNF co-treatment in primary muscle myoblast cultures. Primary myoblasts were maintained in myogenic cell medium on collagen-coated plastic dishes. Myoblasts were pretreated with DMSO carrier control (A-B) or 10 μM SB (C) for one-hour and then were stimulated with anisomycin (1 μg/ml) and TNF (50 ng/ml) (B-C) or mock treatment (A) for 30 minutes. Cells were collected, fixed, permeabilized, stained, and analyzed for phospho-MK2 positivity by flow cytometry.

FIG. 12. p21, Myogenin, and Pax7 expression in freshly isolated and hydrogel-cultured

MuSCs from young and old mice. (A-C) Real-time PCR gene expression analysis of the cell cycle inhibitor p21 (A), the myogenic commitment marker Myogenin (B), and the muscle stem cell marker Pax7 (C), in young and old MuSCs either freshly isolated or after one week culture on soft (12 kPa) hydrogels. Transcript levels of each gene normalized to Gapdh are plotted relative to freshly isolated young MuSCs for each condition (mean±s.e.m. from n=3 independent experiments; P<0.05 by t test). (D-F) p38 inhibition prevents myogenin protein expression in hydrogel-cultured MuSCs. MuSCs isolated from young mice were cultured on soft hydrogels with DMSO carrier control or 10 μM SB202190. After one week, cells were fixed, permeabilized, and stained with an anti-myogenin primary antibody and a fluorescently-conjugated secondary antibody (D, right panels). Nuclei were counter-stained with Topro3 (D, left panels). Cells (n>250 per condition) were imaged by confocal microscopy. Images were segmented into nuclear regions and anti-myogenin fluorescence was integrated within each region using MetaMorph software. (E) Myogenin-positivity was determined from a histogram of cellular myogenin intensities. Examples of myogenin+ cells are marked by arrowheads in (D). (F) Bar graph indicates the percentage of myogenin+ cells was greatly diminished in SB-treated MuSCs compared to control MuSCs. P<0.05 by Fisher's exact test.

FIG. 13. A second p38 inhibitor SB203580 improves the transplant engraftment efficiency of young MuSCs cultured for one week. MuSCs isolated from young GFP/Luciferase mice were cultured on soft (12 kPa) hydrogels and exposed to DMSO carrier control (−), 10 μM SB202190, or 10 μM SB203580. After one week, 100 cells from each condition were transplanted into recipient mice and subsequently assayed for engraftment after one-month by BLI. Graph depicts the percentage of mice from each transplantation condition (n=44 for DMSO and SB202190; n=10 for SB203580) that displayed a BLI value consistent with engraftment. P<0.05 by Fisher's exact test.

FIG. 14. Effect of p38 inhibition on Pax7 expression and transplantation function of MuSCs cultured on rigid hydrogel substrates. (A) Expression of the muscle stem cell marker Pax7, as assessed by real-time PCR, in young and old MuSCs cultured on rigid (˜10⁶ kPa) or soft (12 kPa) hydrogels exposed to DMSO carrier control (−) or 10 μM SB202190 (SB) for one-week. Transcript levels normalized to Gapdh for each condition and plotted relative to a freshly isolated young MuSC control sample are shown (mean±s.e.m. from n=3 independent replicates; P<0.05 by t test). (B) p38 inhibition does not support significant improvement of function of young and old MuSCs cultured on rigid hydrogels. MuSCs isolated from young and old GFP/Luciferase mice were cultured on rigid hydrogels and exposed to DMSO carrier control (−) or 10 μM SB202190 (SB). After one week, 100 cells from each condition were transplanted into recipient mice and were assayed one-month later for engraftment by BLI. Scatter graph of BLI signals from multiple transplants (n=18 for young MuSCs; n=10 for old MuSCs) across two independent experiments. Engraftment threshold indicated by horizontal dashed line. Percentages of mice in each transplantation condition that engrafted above threshold are reported in the inset text. No comparisons were statistically significant (n.s.) by Fishers exact test.

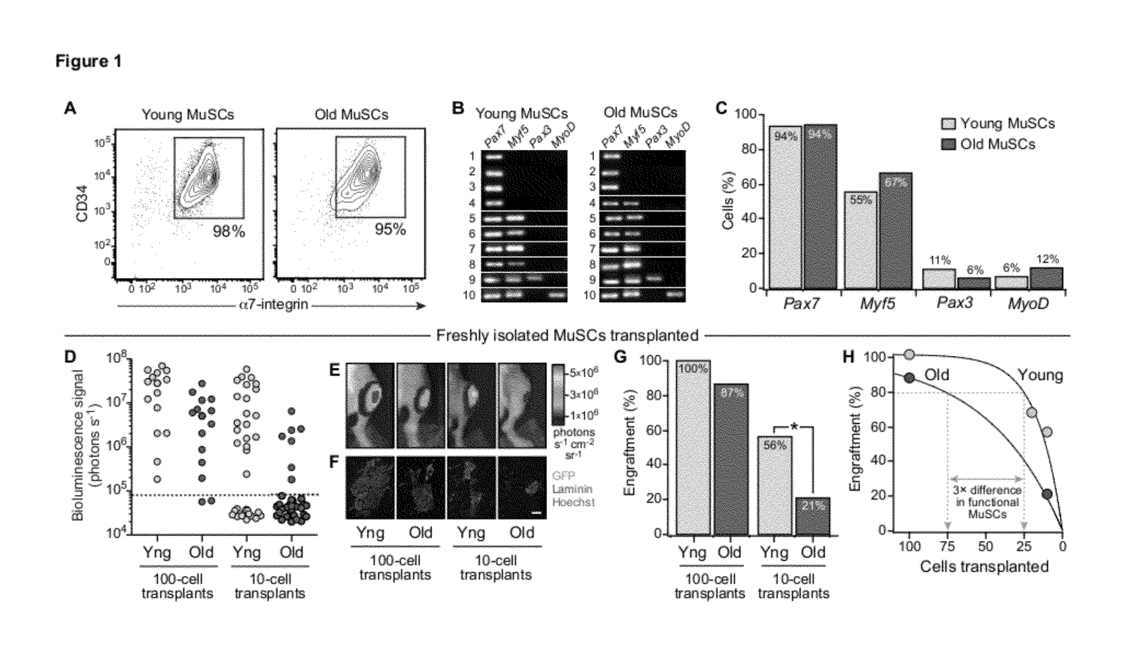

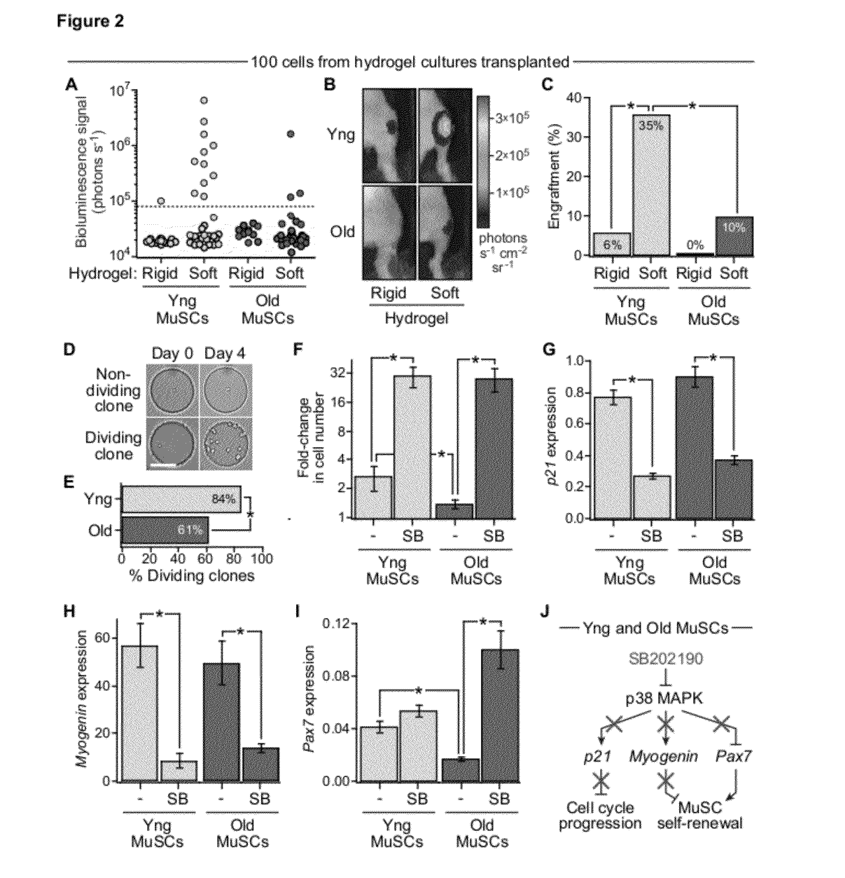

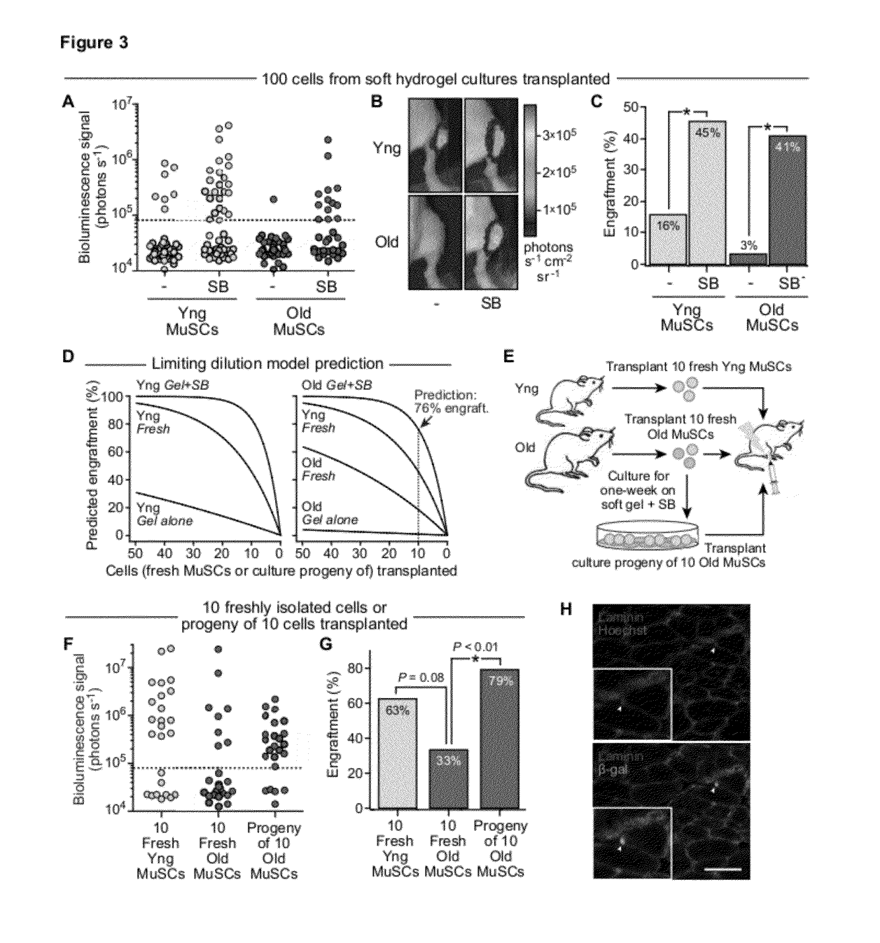

FIG. 15. Limiting dilution analysis of MuSC transplant engraftment indicates expansion of functional stem cells in p38-inhibited hydrogel cultures. (A-B) Engraftment percentages of young (A) and old (B) MuSCs transplanted in varying cell numbers after isolation (fresh) or one-week culture on soft (12 kPa) hydrogels±10 μM SB202190 (SB) treatment. Symbols are experimental data; lines are model fits to equation 1. Data is summarized from experiments presented in FIGS. 1E-G, 2A-C, and 3A-C. (C) Data from each condition were fit to an exponential limiting dilution model, relating the engraftment percentages to the number of cells transplanted. Exponential parameter (03) and model fitness (R²) values for each experimental condition are tabulated. Limiting dilution model equations were used to derive an estimate for the effective number of functional MuSCs with engraftment potential (defined as freshly isolated young MuSCs) within any number of total cells from any condition. The number of functional MuSCs present in the progeny of any number of MuSCs (e.g. 10 cells) initially seeded in the cell culture experiments can be predicted (equation 2) based on the measured cell proliferation over one week of culture (see FIG. 2F). Similarly, the transplant engraftment percentage of the cell culture progeny from any starting MuSC cell number can be predicted (equation 3) using the measured cell proliferation data and the exponential model parameter (see FIG. 3D).

FIG. 16. Transplantation of SB-treated old MuSCs leads to slightly reduced regenerative extent compared to freshly isolated young or old MuSCs. Ten young or old freshly isolated MuSCs and the progeny of 10 old MuSCs (˜300 cells total) after one week on soft hydrogels with SB treatment were transplanted into recipient mice (see FIG. 3E-G). (A) Representative BLI images one month post-transplant. (B) Representative immunofluorescence images of transverse sections of recipient muscles at one month post-transplant. GFP, green; Laminin, red; Hoechst, blue. Scale bar, 100 μm (C) Box-and-whisker plots representing the median (line), 50% confidence interval (box), and 95% confidence interval (whiskers) of BLI values from all engrafted transplants from each condition. Dashed line represents the engraftment threshold. P<0.05 by Mann-Whitney test for both fresh MuSC sources compared to the old MuSC progeny.

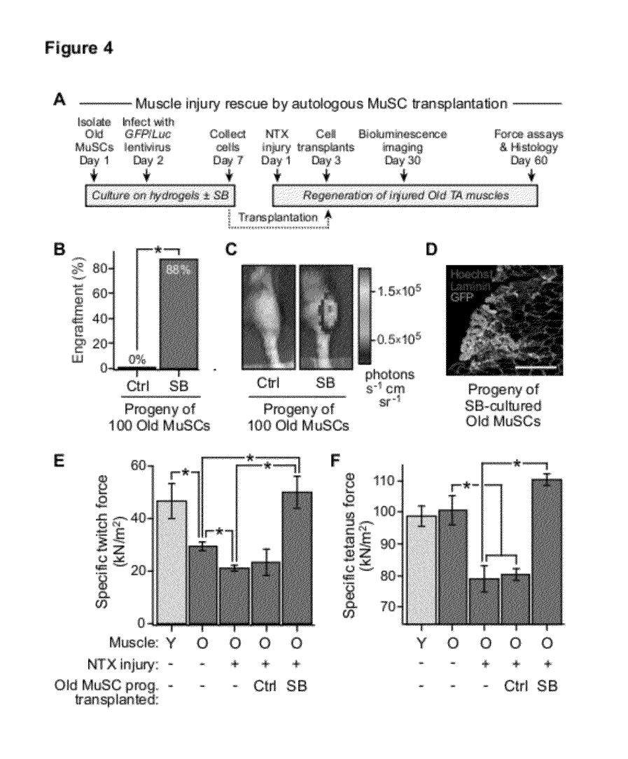

FIG. 17. Engraftment of the culture progeny of old MuSCs from control or SB-treated soft hydrogel cultures following transplantation into syngeneic old mice. Old C57BL/6 MuSCs cultured on soft hydrogels without (Ctrl; ˜200 cells total) and with 10 μM SB202190 (SB; ˜3000 cells total) were infected with a GFP/Luciferase lentivirus and then the progeny of 100 cells were transplanted in into old syngeneic muscles (see also FIG. 4A-D). Recipient old muscles were acutely injured by notexin injection two days prior to transplantation. Bioluminescence imaging signals from multiple transplants (n=8) at one month post-transplant (dashed line indicates detection threshold). Percentage of transplants that engrafted above threshold are reported in the insets. P<0.05 by Fisher's exact test.

FIG. 18. Muscle contractile force measurements following cultured MuSC transplantations. (A-D). Muscle contractile twitch and tetanic forces were assessed in uninjured control tibialis anterior muscles in young (Y) or old (O) mice and at two months post-notexin (NTX) injury in old mice (see FIG. 4). Some NTX-injured muscles were transplanted with the progeny of 100 old MuSCs that were cultured for one week on soft hydrogels without (Ctrl) or with SB treatment. Five replicate twitch and tetanus force measurements were performed in sequence on each muscle and were averaged to report a value for each muscle. Muscles from multiple mice were averaged to report a value for each condition. (A) Representative twitch force traces. Time is relative to the start of muscle excitation. Resting (pre-stimulation) tension force is subtracted out. (B) Mean±s.e.m. twitch forces from n=3-5 muscles per condition. P<0.05 by t test. (C) Representative tetanus force traces. Time is relative to the start of muscle excitation. Resting (pre-stimulation) tension force is subtracted out. (D) Mean±s.e.m. tetanus forces from n=3-5 muscles per condition. P<0.05 by t test. (E) Physiological cross-sectional area (PCSA) values from each condition. The PCSA of assayed tibialis anterior muscles was calculated by relating muscle volume, fiber length, and pennation angle of the muscle fibers, as described above. Mean±s.e.m. from n=3-5 muscles per condition. No comparisons were significant by t test. These PCSA values were used to calculate specific forces reported in FIG. 4E-F.

FIG. 19. Muscle contractile force measurements following systemic SB202 administration. (A-C). Muscle contractile twitch and tetanic forces were assessed in uninjured control tibialis anterior muscles in young (Y, 2 months) or old (O, 24 months). Muscles from old mice were assayed at two months post-notexin (NTX) injury (or saline [−] control). Some NTX-injured old mice were systemically administered SB202 (5 mg/kg body weight in 0.9% NaCl/H₂O, five daily doses by IP injection starting at one day after NTX injury). Five replicate twitch and tetanus force measurements were performed in sequence on each muscle and were averaged to report a value for each muscle. Muscles from multiple mice were averaged to report a value for each condition. (B) Mean±s.e.m. specific twitch forces from n=3-5 muscles per condition. P<0.05 by t test. Physiological cross-sectional area (PCSA) values not shown. (C) Mean ±s.e.m. specific tetanus forces from n=3-5 muscles per condition. P<0.05 by t test.

### DESCRIPTION OF THE SPECIFIC EMBODIMENTS

Methods and compositions are provided for rejuvenation and proliferation of stem and/or progenitor cells having an intrinsic loss of capability due to an aging process. By administering agents that inhibit the p38 MAPK signaling pathway, stem cells in tissues are induced to proliferate, thereby regenerating the tissue by providing appropriate numbers of cells that differentiate into the tissue of interest.

A number of somatic stem/progenitor cells are known in the art, and benefit from the methods of the invention. These cells include satellite cells in skeletal muscle; hematopoietic stem cells; mesenchymal stem cells; neural stem cells; melanocytes, epidermal stem cells, intestinal stem cells, cardiomyocytes, and the like.

For example, it is shown herein that muscle stem cells from an aged individual maintained on a soft hydrogel with elasticity similar to muscle tissue in the presence of a p38 inhibitor expand in culture. In such conditions transcription of the cell cycle inhibitor p21, and the commitment marker Myogenin is decreased in the aged stem cells, while there is increased transcription of the MuSC transcription factor Pax7. The p38 inhibition enhances cell proliferation and induces a stem cell-specific gene expression profile in aged muscle stem cells, where the regenerative capacity of both young and old MuSCs is improved to similarly high levels.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims.

As used herein the singular forms “a”, “an”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

Tissue rejuvenating agent. As used herein and as demonstrated in the Examples, agents that rejuvenate aged stem cells are agents that inhibit the p38 MAPK signaling pathway. The MAPKs operate as a series of kinase modules beginning with the MAPK kinase kinases (MKKKs), which phosphorylate MAPK kinases (MKKs), which ultimately phosphorylate MAP kinases (MAPK). The p38 family has four members: α, β, γ and δ. p38α may also be referred to as MAPK14 (mitogen-activated protein kinase 14), CSBP, or SAPK2A (GeneID: 1432; Uniprot Q16539).

P38 upregulates cytokine production by several mechanisms such as direct phosphorylation of transcription factors such as AP-1, or by stabilization and increased translation of mRNAs containing 3′ untranslated region AU-rich elements (AREs) by phosphorylation of ARE binding proteins. Inhibitors of interest generally target p38 MAPK itself, although inhibitors of the upstream members of the signaling pathway, MAPK kinase kinase (MLK3, TAK, DLK) and MAPK kinase (MKK 3/6) are also of interest, as are the downstream effectors of the signaling pathway.

Inhibition of p38 MAPK signaling may utilize various agents, including a number of small molecule inhibitors, members of which are known in the art, inhibitory polynucleotides such as RNAi, anti-sense oligonucleotides; and the like. See, for example, Schindler et al. (2007) J. Dental Res. 86:800; Kumar et al. (2003) Nature Reviews 2:717; and Zheng et al. (2007) Trends in Pharmacological Sciences 28:286, each herein specifically incorporated by reference.

Included, without limitation, as small molecule inhibitors are those known in the art. Inhibitors may be selective for certain p38 MAPK, e.g. alpha and beta, or may be broadly inhibitory of the protein family. Included and of particular interest are SB202190; SB 203580; VX-745 which are potent inhibitors of p38α and β but not γ or δ.

p38 MAPK inhibitors that have undergone clinical trials include GSK-681323 (GlaxoSmithKline); SB-85635; AMG-548 (Amgen); AVE-9940 (Sanofi-Aventis); PS-540446 (Pharmacopoeia); PS-516895 (Bristol Myers Squibb); SCIO-469 (Scios); SC80036 (Pfizer); PH-797804; BIRB-796 (Boehringer Ingelheim); VX-745 (Vertex); VX-702; R04402257 (Roche); RWJ 67657 (Johnson & Johnson); TAK-715 (Takeda).

Classes of inhibitors include non-diaryl heterocycle compounds (see Cirillo et al. (2002) Curr. Top. Med. Chem. 2(9):1021-1035); imidazole-based and pyrido-pyrimidin-2-one compounds (see Natarajan et al. (2005) Curr. Top. Med. Chem. 5(10):987-1003); anti-oxidants (see Sadowska et al. (2007) Pulm Pharmacol Ther. 20(1):9-22); next generation inhibitors (see Zhang et al. (2007) Trends Pharmacol Sci. 28(6):286-95), each herein specifically incorporated by reference. Other inhibitors of the pathway may target inflammatory cytokines that upregulate p38 activation such as TNF, IL-1 and others (see Silva et al. (2010) Immunotherapy 2(6):817-833; Furst et al. (2005) Ann Rheum Dis. 64 Suppl 4:iv2-14); antisense and interfering oligonucleotides; activators of/ecotopic expression of protein phosphatases that de-phosphorylate p38 (e.g. mitogen-activated protein kinase phosphatase-7); expression of dominant-negative forms of the upstream adapters in the p38 pathway (e.g. dominant negative MKK3 or MKK6 or ASK1); and the like. p38 inhibitors can be small molecules, siRNA (e.g., US2005/0239731; WO 04/097020; WO 03/072590), antisense molecules, proteins, ribozymes or antibodies.

Some specific examples of p38 inhibitors that can be used in the present invention, without being limited thereto, are selected in the group consisting of AMG 548 (Amgen); ARQ 101 (Arqule); ARRY-371797, ARRY-614, and AR00182263 (ARRAY BIO-PHARMA); AZD6703 (Astrazeneca); RPR200765A and RPR203494 (Aventis); BIRB796 (Boehringer Ingelheim); SB242235, SB239063 and SB681323 (Glaxosmithkline); R04402257 and R03201195 (Hoffman-Laroche); RWJ67657, RWJ67671, RWJ67568, RWJ67411, RWJ66430 (Johnson & Johnson Pharmaceutical); KC706 (Kemia); L-167307 (Merck); SC-80036 (Pfizer); SC10-469, SC10-323, SD-282 and SCI-496 (Scios); TAK715 (Takeda); VX-702, VX-850, and VX-745 (Vertex); and SB202190, SB203580, SB220025, SB239063, SC68376, SKF-86002, Compound 37 (Amgen's 657417), SX-011, P38 MAPKinase inhibitor III (ref cat No 506121) and P38 MAPKinase inhibitor (cat No 506126) (Calbiochem). Other p38 inhibitors are described in the following patents and patent applications U.S. Pat. No. 5,716,972, U.S. Pat. No. 5,686,455, U.S. Pat. No. 5,656,644, U.S. Pat. No. 5,593,992, U.S. Pat. No. 5,593,991, U.S. Pat. No. 5,663,334, U.S. Pat. No. 5,670,527, U.S. Pat. Nos. 5,559,137, 5,658,903, U.S. Pat. No. 5,739,143, U.S. Pat. No. 5,756,499, U.S. Pat. No. 6,277,989, U.S. Pat. No. 6,340,685, U.S. Pat. No. 5,716,955, U.S. Pat. No. 7,071,198, WO 92/12154, WO 94/19350, WO 95/09853, WO 95/09851, WO 95/09847, WO 95/09852, WO 97/25048, WO 97/25047, WO 97/33883, WO 97/35856, WO 97/35855, WO 97/36587, WO 97/47618, WO 97/16442, WO 97/16441, WO 97/12876, WO 98/25619, WO 98/06715, WO 98/07425, WO 98/28292, WO 98/56377, WO 98/07966, WO 98/56377, WO 98/22109, WO 98/24782, WO 98/24780, WO 98/22457, WO 98/52558, WO 98/52559, WO 98/52941, WO 98/52937, WO 98/52940, WO 98/56788, WO 98/27098, WO 98/47892, WO 98/47899, WO 98/50356, WO 98/32733, WO 99/58523, WO 99/01452, WO 99/01131, WO 99/01130, WO 99/01136, WO 99/17776, WO 99/32121, WO 99/58502, WO 99/58523, WO 99/57101, WO 99/61426, WO 99/59960, WO99/59959, WO 99/00357, WO 99/03837, WO 99/01441, WO99/01449, WO 99/03484, WO 99/15164, WO 99/32110, WO99/32111, WO 99/32463, WO 99/64400, WO 99/43680, WO 99/17204, WO 99/25717, WO 99/50238, WO 99/61437, WO99/61440, WO 00/26209, WO 00/18738, WO 00/17175, WO00/20402, WO 00/01688, WO 00/07980, WO 00/07991, WO00/06563, WO 00/12074, WO 00/12497, WO 00/31072, WO00/31063, WO 00/23072, WO 00/31065, WO 00/35911, WO 00/39116, WO 00/43384, WO 00/41698, WO 00/69848, WO00/26209, WO 00/63204, WO 00/07985, WO 00/59904, WO00/71535, WO 00/10563, WO 00/25791, WO 00/55152, WO00/55139, WO 00/17204, WO 00/36096, WO 00/55120, WO00/55153, WO 00/56738, WO 01/21591, WO 01/29041, WO 01/29042, WO 01/62731, WO 01/05744, WO 01/05745, WO01/05746, WO 01/05749, WO 01/05751, WO 01/27315, WO01/42189, WO 01/00208, WO 01/42241, WO 01/34605, WO01/47897, WO 01/64676, WO 01/37837, WO 01/38312, WO01/38313, WO 01/36403, WO 01/38314, WO 01/47921, WO 01/27089, WO0246158, WO03002542, WO03097062, WO03103590, WO03099796, WO03084539, WO03097062, WO03104223, WO04099156, WO04110990, WO04092144, WO04022712, WO04016267, WO04113348, WO04004725, WO04032874, WO04076450, WO04020440, WO04078116, US2004092547, WO04020438, WO04014900, WO04014920, WO04091625, WO05077945, WO05100338, WO05061486, WO05060967, WO05105743, WO05000405, WO05073232, WO05073189, WO05073217, WO05073219, WO05033102, WO05012241, WO05030151, WO05105790, WO05032551, WO05075478, WO05076990, WO05009973, WO05075425, WO05000298, WO05080380, WO05009367, WO05033086, WO05034869, WO05033072, WO05042540, WO05082862, WO05063766, WO05120509, WO05097758, WO06127678, WO06118914, WO06071456, US2006234911, WO06055302, EP1707205, WO06099495, WO06116355, WO06122230, WO06084017, WO06067168, WO06104915, WO06062982, WO06112828, WO06028836, WO06018842, WO06047354, WO06020904, WO06040056, WO06006691, WO06009741, WO06044869, WO06018727, WO06018735, WO06052810, US2006058296, WO06010082, WO06003517, WO06020365, US2006079461, WO06104889, WO06018718, WO06128268, WO06110173, WO06039718, WO06040666, WO06040649, WO06013095, US2006052390, WO07015877, WO07015866, WO07000340, WO07000339, WO07000337, WO07016392, DE 19842833, and JP 2000 86657, whose disclosures are all incorporated herein by reference in their entirety.

In some embodiments the inhibitor is selective for the alpha, or the alpha/beta form of p38, for example where the IC₅₀ of the inhibitor is at least 5, 10, 50, 100, 500, 1000, 5000 or 10000 fold greater for p38 isoforms γ and δ in comparison with the IC₅₀ for p38α.

The therapeutically effective amount of p38 pathway inhibitor varies depending upon the administration mode, the age, body weight, sex and general health of the subject. It will be appreciated that there will be many ways known in the art to determine the therapeutically effective amount for a given application. The appropriate dose can be from 0.01 to 1000 mg/day. The therapeutically effective amount of p38 pathway inhibitors can be administered once, twice, thrice, or four times a day. Alternatively, the therapeutically effective amount of p38 pathway inhibitors can be administered every day, every two day, or one, two, or three times a week.

The p38 pathway inhibitor may be administered orally, parenterally, by inhalation spray, topically, rectally, nasally, buccally, vaginally or via an implanted reservoir. The term “parenteral” as used herein includes subcutaneous, intravenous, intramuscular, intra-tumoral, intra-articular, intra-synovial, intrasternal, intrathecal, intrahepatic, and intralesional. Preferably, the inhibitor is administered as a depot, where the drug is substantially retained in the tissue of interest. The p38 pathway inhibitor can be formulated as a pharmaceutical composition generally comprising a pharmaceutically acceptable carrier. By a pharmaceutically acceptable carrier is intended a carrier that is physiologically acceptable to the treated mammal while retaining the therapeutic properties of the drug with which it is administered. For example, a pharmaceutically acceptable carrier can be physiological saline solution. Other pharmaceutically acceptable carriers are known to one skilled in the art and described for instance in Remington: The Science and Practice of Pharmacy (20.sup.th ed., ed. A. R. Gennaro A R., 2000, Lippincott Williams & Wilkins).

A determination of effective dose, and effective combination of agents may be determined empirically, for example using animal models as provided herein. In vitro models are also useful for the assessment of dose and selection of agent. For example, cultures are described herein where the regenerative potential of stem cells are evaluated in the absence or presence of serum from an individual where stem cell regeneration is comprised relative to a healthy young individual. Such cultures may be used to assay for the effectiveness of agents alone, or in combinations.

Aged. As used herein, the term aged refers to the effects or the characteristics of increasing age, particularly with respect to the diminished ability of somatic tissues to regenerate in response to damage, disease, and normal use. One measure of aging, therefore, is evidenced by the intrinsic lack of ability of somatic stem cells to regenerate tissues. Alternatively, aging may be defined in terms of general physiological characteristics. The rate of aging is very species specific, where a human may be aged at about 50 years; and a rodent at about 2 years. In general terms, a natural progressive decline in body systems starts in early adulthood, but it becomes most evident several decades later. One arbitrary way to define old age more precisely in humans is to say that it begins at conventional retirement age, around about 60, around about 65 years of age. Another definition sets parameters for aging coincident with the loss of reproductive ability, which is around about age 45, more usually around about 50 in humans, but will, however, vary with the individual.

The term “cell culture” or “culture” means the maintenance of cells in an artificial, in vitro environment. Culture conditions may include, without limitation, a specifically dimensioned container, e.g. flask, roller bottle, plate, 96 well plate, etc.; culture medium comprising suitable factors and nutrients for growth of the desired cell type; and a substrate on the surface of the container or on particles suspended in the culture medium. By “container” is meant a glass, plastic, or metal vessel that can provide an aseptic environment for culturing cells.

A “long term culture” used herein refers to a culture in which stem cells grow, differentiate and are viable for at least a few hours, at least about 7 days, at least about 14 days, at least about 21 days, or more.

The terms “primary culture” and “primary cells” refer to cells derived from intact or dissociated tissues or organ fragments. A culture is considered primary until it is passaged (or subcultured) after which it is termed a “cell line” or a “cell strain.” The term “cell line” does not imply homogeneity or the degree to which a culture has been characterized. A cell line is termed “clonal cell line” or “clone” if it is derived from a single cell in a population of cultured cells. Primary cells can be obtained directly from a human or animal adult or fetal tissue, including blood. The primary cells may comprise a primary cell line, or such as, but not limited to, a population of muscle satellite cells.

Substrate. As used herein, a substrate refers to a coating of a semi-solid matrix on a surface contacted by cells in a culture condition, where the surface may include, without limitation, the bottom of plates and flasks, etc., the inner wall of a roller bottle; or the surface of rods, particles, filaments and the like present within a culture container. For the methods of the present invention, a substrate is sufficiently thick that it masks the physical properties of the container, usually at least about 50 μm thick, at least about 100 μm thick, at least about 1000 μm. There are generally no adverse effects associated with increased thickness, but for convenience it may be desirable to have a substrate not more than about 10 mm in thickness.

In the methods of the invention, the elasticity of the substrate is selected to have physiological parameters, for example being selected to have similar properties to the elasticity of the tissue from which the stem cell derived. Elasticity may be measured by any convenient method, as is known in the art. For example see, inter alia, Kaletunc et al. (1991) Food Hydrocolloids 5:237-247; Krall and Weitz (1998) Physical Review Letters 80:778-781; Melekaslan et al. (2003) Polymer Bulletin 50:287-294; each herein incorporated by reference. Conveniently a shear rheometer may be used, as described in the examples.

It will be appreciated by one of skill in the art that the elasticity of the substrate will vary, depending on the specific somatic stem cell that is being cultured. For example, it is found the muscle stem cells are optimally grown on a substrate of at least about 1 kPa and not more than about 1000 kPa, usually at least about 5 kPa and not more than about 100 kPa. Similar elasticity may be utilized for stem cells residing in other soft tissue niches, e.g. neural stem cells, hematopoietic stem cells, liver stem cells, etc. Hard tissues, such as bone, may have a more rigid structure, e.g. at least about 100 kPa and up to as much as 10⁶ kPa.

Preferred substrates for the methods of the invention are hydrogels. The term “hydrogel” as used herein refers to a network of polymer chains that are water-insoluble, sometimes found as a colloidal gel in which water is the dispersion medium. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published US patent application 20100055733, herein specifically incorporated by reference. The cell culture platform of the present disclosure may be fabricated from a soft and inert substrate that imbibes large amounts of water, thus approximating critical physicochemical aspects of the stem cell niche.

The term “polymeric composition” as used herein refers to a single compound species or a mixture of compound species that may be cross-linked to form a polymer. Such precursor compounds include, but are not limited to, such as poly(ethylene glycol), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, alginate, protein polymers, methylcellulose and the like. The polymer compounds before polymerization may be toxic to, or otherwise inhibit the proliferation of a vertebrate cell, but it will be understood by those in the art that when polymerized, the polymer will be inert with respect to any cell or cell line in contact with the polymer.

In embodiments of the invention, the hydrogel composition may be selected from the group consisting of: a poly(ethylene glycol), a polyaliphatic polyurethane, a polyether polyurethane, a polyester polyurethane, a polyethylene copolymer, a polyamide, a polyvinyl alcohol, a polypropylene glycol, a polytetramethylene oxide, a polyvinyl pyrrolidone, a polyacrylamide, a poly(hydroxyethyl acrylate), and a poly(hydroxyethyl methacrylate).

The elasticity of the substrate may be influenced by a variety of factors, including the branching of the polymer, the concentration of polymer, and the degree of cross-linking. For example, where the polymer is formed of PEG-VS, the length of the PEG monomer and the branching, e.g. 2 arm, 4 arm, 8-arm, and the like may be varied to achieve the desired elasticity. Optionally a non-swelling hydrogel is used.

Hydrogels of the invention optionally comprise at least one structural protein associated with a stem cell niche, e.g. fibronectin, laminin, collagen, and the like. Alternatively, or in combination, other proteins that have a beneficial or desired effect on the stem cells may be included in the hydrogel. Proteins may be conjugated to the hydrogel through a linker. The term “tether” or “linker” may refer to a molecular structure that conjugates a protein or polypeptide to the hydrogel. It is contemplated that a linker molecule suitable to link a biomolecule to the hydrogels of the disclosure can be, but is not limited to, a maleimide PEG-SVA linker; a dicarboxylic acid that further includes at least one available group, such as an amine group, for conjugating to a prosthetic group; and the like. It is also contemplated that other functional side groups may substitute for the amine group to allow for the linking to selected peptides. Exemplary dicarboxylic acids include, but are not limited to, aspartate, glutamate, and the like, and can have the general formula (HOOC)——(CH₂)ₙ——(CHNH₂⁺)——(CH₂)ₘ——(COOH), where n and m are each independently 0, or an integer from 1 to about 10. It is further considered within the scope of the disclosure for the linker to be a multimer, or a combination, of at least two such dicarboxylic acids. For example, such linker molecules may include, but are not limited to, (aspartate)=x, (glutamate)_{y}, or a combination thereof, where adjacent amino acids can be joined by peptide bonds, and the like. The subscripts x and y are each independently 0, or an integer from 1 to about 12.

Culture medium: The stem cells are grown in vitro in an appropriate liquid nutrient medium, in the presence of a p38 pathway inhibitor at an effective dose. Generally, the seeding level will be at least about 10 cells/ml, more usually at least about 100 cells/ml and generally not more than about 10⁵ cells/ml, usually not more than about 10⁴ cells/ml. Cells may be cultured singly or in groups.

Various media are commercially available and may be used, including Ex vivo serum free medium; Dulbecco's Modified Eagle Medium (DMEM), RPMI, Iscove's medium, etc. The medium may be supplemented with serum or with defined additives. For example a medium may include 5%, 10%, 15% serum, as known in the art. Appropriate antibiotics to prevent bacterial growth and other additives, such as pyruvate (0.1-5 mM), glutamine (0.5-5 mM), 2-mercaptoethanol (1-10×10⁻⁵ M) may also be included. The medium may be any conventional culture medium, generally supplemented with additives such as iron-saturated transferrin, human serum albumin, soy bean lipids, linoleic acid, cholesterol, alpha thioglycerol, crystalline bovine hemin, etc., that allow for the growth of hematopoietic cells. In some circumstances, proliferative factors that do not induce cellular differentiation may be included in the cultures, e.g. c-kit ligand, LIF, and the like.

Stem cell: The term stem cell is used herein to refer to a mammalian cell that has the ability both to self-renew, and to generate differentiated progeny (see Morrison et al. (1997) Cell 88:287-298). Generally, stem cells also have one or more of the following properties: an ability to undergo asynchronous, or asymmetric replication, that is where the two daughter cells after division can have different phenotypes; extensive self-renewal capacity; capacity for existence in a mitotically quiescent form; and clonal regeneration of all the tissue in which they exist, for example the ability of hematopoietic stem cells to reconstitute all hematopoietic lineages. “Progenitor cells” differ from stem cells in that they typically do not have the extensive self-renewal capacity, and often can only regenerate a subset of the lineages in the tissue from which they derive

Stem cells may be characterized by both the presence of markers associated with specific epitopes identified by antibodies and the absence of certain markers as identified by the lack of binding of specific antibodies. Stem cells may also be identified by functional assays both and in vivo, particularly assays relating to the ability of stem cells to give rise to multiple differentiated progeny.

Somatic Stem cells: Somatic stem cells are resident in differentiated tissue, but retain the properties of self-renewal and ability to give rise to multiple cell types, usually cell types typical of the tissue in which the stem cells are found. Numerous examples of somatic stem cells are known to those of skill in the art, including muscle stem cells (including without limitation satellite cells as described above); hematopoietic stem cells, epithelial stem cells, neural stem cells; mesenchymal stem cells; and the like.

Stem cells of interest include muscle stem cells, which may be evidenced by the ability to engraft and repopulate the myofiber-associated compartment in vivo following intramuscular injection, and subsequent maintenance of myogenic-colony forming capacity. Muscle cells include skeletal, cardiac and smooth muscles, but particularly skeletal muscle.

Stem cells of interest include muscle satellite cells; hematopoietic stem cells and progenitor cells derived therefrom (U.S. Pat. No. 5,061,620); neural stem cells (see Morrison et al. (1999) Cell 96:737-749); embryonic stem cells; mesenchymal stem cells; mesodermal stem cells; liver stem cells, etc.

The cells of interest are typically mammalian, where the term refers to any animal classified as a mammal, including humans, domestic and farm animals, and zoo, laboratory, sports, or pet animals, such as dogs, horses, cats, cows, mice, rats, rabbits, etc. Preferably, the mammal is human.

Hematopoietic stem cells (HSCs) have the ability to renew themselves and to give rise to all lineages of the blood. Conditions of the aged that benefit from activation of HSC include, for example, conditions of blood loss, such as surgery, injury, and the like, where there is a need to increase the number of circulating hematopoietic cells. Anemia is an abnormal reduction in red blood cells, which can occur from a malfunction in the production of red blood cells. Weakness and fatigue are the most common symptoms of even mild anemia. Anemia in the elderly is often due to causes other than diet, particularly gastrointestinal bleeding or blood loss during surgery. Anemia in older people is also often due to chronic diseases and folic acid and other vitamin deficiencies.

Neural stem cells are primarily found in the hippocampus, and may give rise to neurons involved in cognitive function, memory, and the like. Neural stem and progenitor cells can participate in aspects of normal development, including migration along well-established migratory pathways to disseminated CNS regions, differentiation into multiple developmentally- and regionally-appropriate cell types in response to microenvironmental cues, and non-disruptive, non-tumorigenic interspersion with host progenitors and their progeny.

Aged individuals often suffer from a diminution of neural function. As such, these cells find use in the treatment of a variety of conditions, including traumatic injury to the spinal cord, brain, and peripheral nervous system; treatment of degenerative disorders including Alzheimer's disease, Huntington's disease, Parkinson's disease; stroke; and the like.

Alzheimer's disease accounts for half to two thirds of all dementia cases. Other causes of dementia include vascular disease (atherosclerosis of the brain blood vessels); traumatic brain injury; Parkinson's, Huntington's, Creutzfeldt-Jakob, and other diseases. Alzheimer's disease is uncommon below 65 years, occurring in fewer than 5% of people aged 65-70, and then increasing in frequency rapidly, so that by 95 years of age as many as 55% of people are affected.

Older adults are also at risk for neural damage resulting from stroke. Older age is also linked with higher rates of post-stroke dementia. Stroke occurs as a result of blood flow blockage to the brain. A reduction of, or disruption in, blood flow to the brain is the primary cause of a stroke. Blockage for even a short period of time can be disastrous and cause brain damage or even death. Ischemic strokes are the more common type, causing over 80% of all strokes.

Parkinson's disease (PD) is a slowly progressive disorder that affects movement, muscle control, and balance. Most Parkinson's victims are elderly. PD develops as cells are destroyed in certain parts of the brain stem, particularly the crescent-shaped cell mass known as the substantia nigra. Nerve cells in the substantia nigra send out fibers to the corpus stratia, gray and white bands of tissue located in both sides of the brain. There the cells release dopamine, an essential neurotransmitter (a chemical messenger in the brain). Loss of dopamine in the corpus stratia is the primary defect in Parkinson's disease.

Stem cells may also be present in the epidermis, giving rise both to epidermal and mesenchymal tissues. Like all the body's tissues, the skin undergoes many changes in the course of the normal aging process. The cells divide more slowly, and the inner layer of the dermis starts to thin. Fat cells beneath the dermis begin to atrophy. In addition, the ability of the skin to repair itself diminishes with age, so wounds are slower to heal. The thinning skin becomes vulnerable to injuries and damage. The underlying network of elastin and collagen fibers, which provides scaffolding for the surface skin layers, loosens and unravels. Skin then loses its elasticity. When pressed, it no longer springs back to its initial position but instead sags and forms furrows. The skin is more fragile and may bruise or tear easily and take longer to heal.

In response to damage of aged skin, for cosmetic purposes, following trauma such as burns, abrasions, etc., it is beneficial to stimulate activation of stem cells. Activating agents may be administered topically, e.g. in combination with agents to enhance penetration through the dermal layers, systemically, using implants, etc.

In many clinical situations, the bone healing condition are less ideal due to decreased activity of bone forming cells, e.g. within aged people. Within bone marrow stroma there exists a subset of non-hematopoietic cells capable of giving rise to multiple cell lineages. These cells termed as mesenchymal stem cells (MSC) have potential to differentiate to lineages of mesenchymal tissues including bone, cartilage, fat, tendon, muscle, and marrow stroma.

A variety of bone and cartilage disorders affect aged individuals. Such tissues are normally regenerated by mesenchymal stem cells. Included in such conditions is osteoarthritis. Osteoarthritis occurs in the joints of the body as an expression of “wear-and-tear”. Thus athletes or overweight individuals develop osteoarthritis in large joints (knees, shoulders, hips) due to loss or damage of cartilage. This hard, smooth cushion that covers the bony joint surfaces is composed primarily of collagen, the structural protein in the body, which forms a mesh to give support and flexibility to the joint. When cartilage is damaged and lost, the bone surfaces undergo abnormal changes. There is some inflammation, but not as much as is seen with other types of arthritis. Nevertheless, osteoarthritis is responsible for considerable pain and disability in older persons.

In conditions of the aged where repair of mesenchymal tissues is decreased, or there is a large injury to mesenchymal tissues, the stem cell activity may be enhanced by administration of tissue regenerating agent(s).

The term “muscle cell” as used herein refers to any cell which contributes to muscle tissue. Myoblasts, satellite cells, myotubes, and myofibril tissues are all included in the term “muscle cells”. Muscle cell effects may be induced within skeletal, cardiac and smooth muscles. Muscle tissue in adult vertebrates will regenerate from reserve myoblasts called “satellite cells”. or mesangioblasts, bone marrow derived cells, muscle interstitial cells, mesenchymal stem cells, etc. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

Various methods may be used to enrich for muscle stem cells, or a complex population comprising muscle stem cells may be cultures. One example of muscle stem cells is cells characterized as CD45⁻, CD11b⁻, CD31⁻, Sca1^{-b , α7} integrin+, and CD34⁺. Various other criteria have been used to select for muscle stem cells, including without limitation: enrichment for (CD11b/CD31/Sca1/CD45)− CD34+ alpha7integrin+ (see Sacco et al. (2008) Nature 456(7221):502-506); enrichment for (Sca1/Mac1/CD45)−CD29+CXCR4+ (see Cerletti et al. (2008) Cell 134(1):37-47); enrichment for Syndecan3+/Syndecan4+/ABCG2-/Sca− (see Tanaka et al. (2009) Cell Stem Cell. 4(3):217-25); use of whole single muscle fibers (see Collins et al. (2005) Cell 122(2):289-301); enrichment for lineage-negative alpha7integrin+ beta1integrin+ cells (see Kuang et al. (2007) Cell 129(5):999-1010); enrichment with SMC2.6 antibody (see Fukada et al. (2004) Exp Cell Res. 296(2):245-55); enrichment from pericytes of CD56+(NCAM) (see Meng et al. (2011) PLoS One. 6(3):e17454); and enrichment for CD34− CD56+ cells (see Pisani et al. (2010) Stem Cells 28(4):753-64); each reference herein specifically incorporated by reference.

In addition to skeletal muscle formation, the regeneration of cardiac muscle in the aging is of interest. For example, following an event such as myocardial infarction; surgery, catheter insertion, atherosclerosis, and the like, cardiac muscle can be damaged. Such damage is not easily repaired in elderly patients, resulting in a loss of function. Administration of p38 MAPK pathway inhibitors following such incidents of muscle damage can increase regeneration of the damaged tissues. The agents may be administered systemically, or using a stent, catheter, implant, and the like that increase the local concentration of the active agent.

Muscle stem cells expanded by the methods of the invention may be implanted into a recipient subject mammal, where the cells or population of cells differentiate into muscle cells. Muscle regeneration as used herein refers to the process by which new muscle fibers form from muscle progenitor cells. A therapeutic composition will usually confer an increase in the number of new fibers by at least 1%, more preferably by at least 20%, and most preferably by at least 50%. The growth of muscle may occur by the increase in the fiber size and/or by increasing the number of fibers. The growth of muscle may be measured by an increase in wet weight, an increase in protein content, an increase in the number of muscle fibers, an increase in muscle fiber diameter; etc. An increase in growth of a muscle fiber can be defined as an increase in the diameter where the diameter is defined as the minor axis of ellipsis of the cross section.

Muscle regeneration may also be monitored by the mitotic index of muscle. For example, cells may be exposed to a labeling agent for a time equivalent to two doubling times. The mitotic index is the fraction of cells in the culture which have labeled nuclei when grown in the presence of a tracer which only incorporates during S phase (i.e., BrdU) and the doubling time is defined as the average S time required for the number of cells in the culture to increase by a factor of two. Alternatively, activation in vivo may be detected by monitoring the appearance of the intermediate filament vimentin by immunological or RNA analysis methods. When vimentin is assayed, a useful activator may cause expression of detectable levels of vimentin in the muscle tissue when the therapeutically useful dosage is provided. Productive muscle regeneration may be also monitored by an increase in muscle strength and agility.

Muscle regeneration may also be measured by quantitation of myogenesis, i.e. fusion of myoblasts to yield myotubes. An effect on myogenesis results in an increase in the fusion of myoblasts and the enablement of the muscle differentiation program. For example, the myogenesis may be measured by the fraction of nuclei present in multinucleated cells in relative to the total number of nuclei present. Myogenesis may also be determined by assaying the number of nuclei per area in myotubes or by measurement of the levels of muscle specific protein by Western analysis.

The survival of muscle fibers may refer to the prevention of loss of muscle fibers as evidenced by necrosis or apoptosis or the prevention of other mechanisms of muscle fiber loss. Muscles can be lost from injury, atrophy, and the like, where atrophy of muscle refers to a significant loss in muscle fiber girth.

The terms “grafting”, “engrafting”, and “transplanting” and “graft” and “transplantation” as used herein refer to the process by which stem cells or other cells according to the present disclosure are delivered to the site where the cells are intended to exhibit a favorable effect, such as repairing damage to a patient's central nervous system, treating autoimmune diseases, treating diabetes, treating neurodegenerative diseases, or treating the effects of nerve, muscle and/or other damage caused by birth defects, stroke, cardiovascular disease, a heart attack or physical injury or trauma or genetic damage or environmental insult to the body, caused by, for example, disease, an accident or other activity. The stem cells or other cells for use in the methods of the present disclosure can also be delivered in a remote area of the body by any mode of administration as described above, relying on cellular migration to the appropriate area in the body to effect transplantation. For example, the term “cell engraftment” as used herein can refer to the process by which cells such as, but not limited to, muscle stem cells, are delivered to, and become incorporated into, a differentiated tissue such as a muscle, and become part of that tissue. For example, muscle stem cells, when delivered to a muscle tissue, may proliferate as stem cells, and/or may bind to skeletal muscle tissue, differentiate into functional myoblasts cells, and subsequently develop into functioning myofibers.

### Tissue Rejuvenation

The methods of the present invention utilize tissue rejuvenating agents that inhibit the p38 MAPK pathway as described above to enhance regeneration of tissues in aged animals. The tissue rejuvenating agents also find use in ex vivo cultures for the expansion of aged stem cells, e.g. expansion on a pliable substrate as described above.

The tissue rejuvenating agent(s) are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration can be achieved in various ways. The agent may be systemic after administration or may be localized by virtue of the formulation, or by the use of an implant that acts to retain the active dose at the site of implantation.

In pharmaceutical dosage forms, the tissue rejuvenating agent(s) and/or other compounds may be administered in the form of their pharmaceutically acceptable salts, or they may also be used alone or in appropriate association, as well as in combination with other pharmaceutically active compounds. The agents may be combined to provide a cocktail of activities. The following methods and excipients are exemplary and are not to be construed as limiting the invention.

Formulations are typically provided in a unit dosage form, where the term “unit dosage form,” refers to physically discrete units suitable as unitary dosages for human subjects, each unit containing a predetermined quantity of active agent in an amount calculated sufficient to produce the desired effect in association with a pharmaceutically acceptable diluent, carrier or vehicle. The specifications for the unit dosage forms of the present invention depend on the particular complex employed and the effect to be achieved, and the pharmacodynamics associated with each complex in the host.

The pharmaceutically acceptable excipients, such as vehicles, adjuvants, carriers or diluents, are commercially available. Moreover, pharmaceutically acceptable auxiliary substances, such as pH adjusting and buffering agents, tonicity adjusting agents, stabilizers, wetting agents and the like, are commercially available. Any compound useful in the methods and compositions of the invention can be provided as a pharmaceutically acceptable base addition salt. “Pharmaceutically acceptable base addition salt” refers to those salts that retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

Those of skill will readily appreciate that dose levels can vary as a function of the specific agent, the severity of the symptoms and the susceptibility of the subject to side effects. Some of the agents will be more potent than others. Preferred dosages for a given agent are readily determinable by those of skill in the art by a variety of means. A preferred means is to measure the physiological potency of a given compound.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease. The therapeutic effect can be measured in terms of clinical outcome or can be determined by immunological or biochemical tests.

For the treatment of disorders in which there is inadequate stem cell activation or there is a rapid deterioration of tissues due to an injury or disease, tissue rejuvenating agent(s) are administered at a dose that is effective to cause an increase of stem cell activation, but which maintains the overall health of the individual. Treatment regimens will often utilize a short-term administration of the active agent; although the treatment may be repeated as necessary. The treatment regime can require administration for prolonged periods, but may be administered as a single dose monthly, semi-monthly, etc. The size of the dose administered must be determined by a physician and will depend on a number of factors, such as the nature and gravity of the disease, the age and state of health of the patient and the patient's tolerance to the drug itself.

In a specific embodiment, the tissue regenerating agent(s) are used for treatment of patients by means of a short-term administration, e.g. of 1, 2, 3 or more days, up to 1 or 2 weeks, in order to obtain a rapid, significant increase in activation. e.g. with daily or semi-daily administration.

For example, a number of conditions relevant to aged populations are characterized by an inability to regenerate tissues. All aged organs and tissues undergo a loss of regeneration and maintenance with age, thus this method is applicable to the aged organ systems in general, including muscle, brain, blood, bones, liver, etc,

Examples of muscular disorders which may be treated include skeletal muscle diseases and disorders; cardiac muscle pathologies; smooth muscle diseases and disorders, etc., particularly Duchene Muscular Dystrophy, cardiomyopathy, atherosclerosis.

A population of cells comprising stem cells can be cultured in vitro on a substrate with a defined elasticity as described above. The cells are maintained in culture for a period of time sufficient to increase the number of assayable stem cells in the culture, or to perform necessary manipulations, genetic or exposure to drugs.

For expansion, the number of assayable stem cells may be demonstrated by a number of assays appropriate to the specific type of stem cell, as described above. Following the initial seeding, there is an expansion, where the number of assayable stem cells having the functional phenotype of the initial cell population can increase from about 2, about 5, to about 100 fold or more. The cells can be frozen using conventional methods at any time, usually after the first week of culture.

After seeding the culture medium, the culture medium is maintained under conventional conditions for growth of mammalian cells, generally about 37° C. and 5% CO₂ in 100% humidified atmosphere. Fresh media may be conveniently replaced, in part, by removing a portion of the media and replacing it with fresh media. Various commercially available systems have been developed for the growth of mammalian cells to provide for removal of adverse metabolic products, replenishment of nutrients, and maintenance of oxygen. By employing these systems, the medium may be maintained as a continuous medium, so that the concentrations of the various ingredients are maintained relatively constant or within a predescribed range. Such systems can provide for enhanced maintenance and growth of the subject cells using the designated media and additives.

Following expansion the cells may be removed from the surface of the substrate by digestion with enzymes, chelators, etc., as known in the art using time, temperature, concentration and selection of reagents that will achieve a partial digestion that leaves aggregates of cells. One of skill in the art can readily perform a simple titration to determine suitable conditions, e.g. using EDTA, elastase; dispase; collagenase; trypsin; blendzyme; and the like.

These cells may find various applications for a wide variety of purposes. The cell populations may be used for screening various additives for their effect on growth and the mature differentiation of the cells. In this manner, compounds which are complementary, agonistic, antagonistic or inactive may be screened, determining the effect of the compound in relationship with one or more of the different cytokines.

The populations may be employed as grafts for transplantation. For example, muscle cells find use in the regeneration or treatment of muscle, hematopoietic cells are used to treat malignancies, bone marrow failure states and congenital metabolic, immunologic and hematologic disorders; and the like.

For therapeutic methods the cells may be used for tissue reconstitution or regeneration in a human patient or other subject in need of such treatment. The cells are administered in a manner that permits them to graft or migrate to the intended tissue site and reconstitute or regenerate the functionally deficient area.

The differentiating cells may be administered in any physiologically acceptable excipient, where the cells may find an appropriate site for regeneration and differentiation. The cells may be introduced by injection, catheter, or the like. The cells may be frozen at liquid nitrogen temperatures and stored for long periods of time, being capable of use on thawing. If frozen, the cells will usually be stored in any suitable medium, for example 10% DMSO, 20% FCS, 70% DMEM medium. Once thawed, the cells may be expanded by use of growth factors and/or feeder cells associated with progenitor cell proliferation and differentiation.

The cells of this invention can be a defined highly enriched FACS sorted population. The cells can be supplied in the form of a pharmaceutical composition, comprising an isotonic excipient prepared under sufficiently sterile conditions for human administration. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells.

The subject methods are useful for both prophylactic and therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues; consequently, the prophylactic therapeutic benefits provided by the invention are also important. Evidence of therapeutic effect may be any diminution in the severity of disease. The therapeutic effect can be measured in terms of clinical outcome or can be determined by immunological or biochemical tests.

The dosage of the therapeutic formulation will vary widely, depending upon the nature of the condition, the frequency of administration, the manner of administration, the clearance of the agent from the host, and the like. The initial dose can be larger, followed by smaller maintenance doses. The dose can be administered as infrequently as weekly or biweekly, or more often fractionated into smaller doses and administered daily, semi-weekly, or otherwise as needed to maintain an effective dosage level.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Experimental

### EXAMPLE 1

### Rejuvenation and Expansion of Muscle Stem Cells from Old Mice

Muscle atrophy in aged individuals is a growing health concern, as the number of people >80 years of age worldwide is rapidly increasing. Currently, there are no systemic or localized pharmacologic or cell-based therapies in clinical use that reverse muscle atrophy in the aged. Muscle stem cells (MuSCs), also known as satellite cells, are essential to skeletal muscle regeneration throughout life. During aging, skeletal muscle mass, strength, and the ability to regenerate after acute injury or immobilization progressively decline, diminishing quality of life. Efforts in many laboratories have focused on identifying mechanisms underlying the regenerative dysfunction of old MuSCs. Accumulating evidence suggests that the aging-related decline in muscle regenerative capacity is due largely to changes in the systemic and local microenvironments. For example, systemic factors from young mice ameliorate muscle regeneration in aged mice following heterochronic parabiosis. In addition, local muscle tissue factors that lead to Wnt accumulation and consequent increased fibrosis or a reduction in Notch activators and diminished satellite cell activation accompany aging and impair regeneration.

Although differences in the inherent capacity of old MuSCs to be activated and proliferate in culture have been reported, old MuSCs are capable of contributing to muscle regeneration following transplantation. Here we investigated the effects of treating FACS-purified MuSCs from old mice with molecules that target pathways associated with MuSC self-renewal or muscle tissue aging. We found that only p38 inhibition, which was previously shown to promote expression of the classical MuSC marker Pax7 and enhance the proliferation of young MuSCs, had a profound impact on old stem cell function. This treatment led not only to increased proliferation and expansion of old muscle stem cells on soft hydrogels, but also to their rejuvenation, so that their absolute numbers and in vivo regenerative function increased to that of freshly isolated young MuSCs. These findings were made possible by using a hydrogel platform that maintains stem cell function ex vivo and a highly sensitive non-invasive imaging assay of regenerative function in mice. Most striking was the finding that transplantation of ex vivo-rejuvenated old muscle stem cells increases strength in damaged muscles of old mice, demonstrating that this strategy can be applied as a localized cell therapy for restoring muscle strength in the elderly.

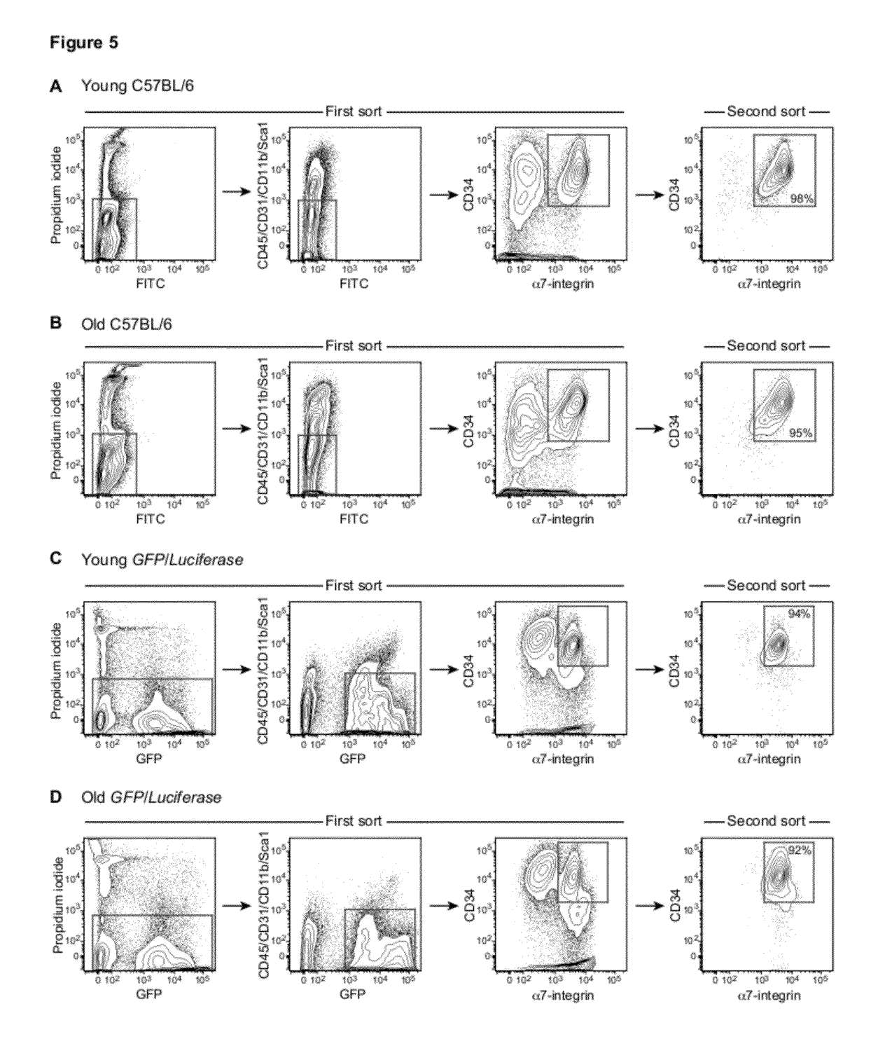

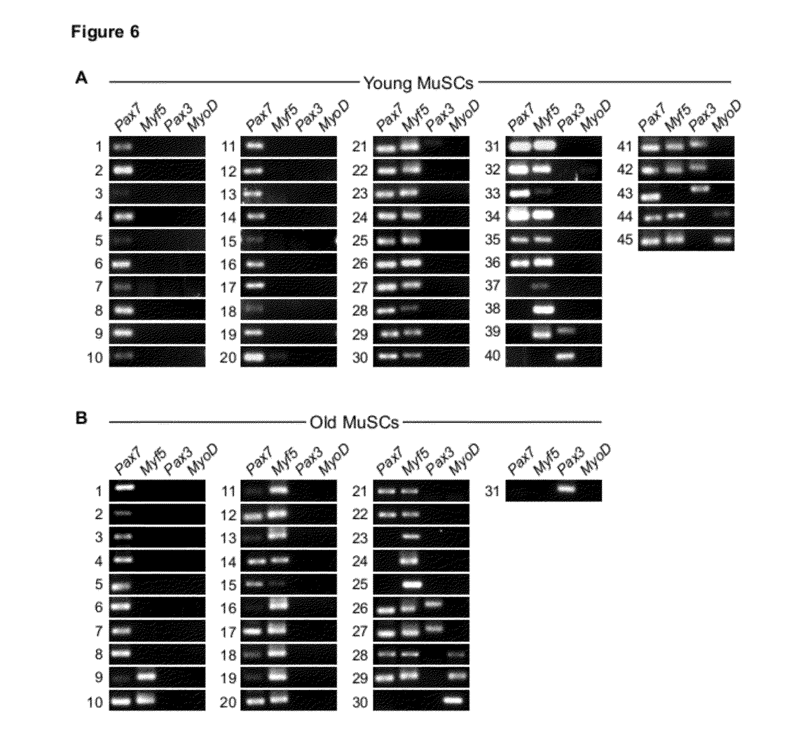

To compare young and old MuSCs, we capitalized on recent advances in MuSC isolation and enrichment. MuSCs were isolated from young and old mice (2 and 24 months, respectively) and enriched to >90% purity using antibodies to CD34 and α7-integrin and fluorescence activated cell sorting (FACS) following enzymatic digestion of hindlimb muscles, as we previously described (FIGS. 1A and 5). Single MuSCs from young and old mice were assayed by nested reverse transcriptase-polymerase chain reaction (RT-PCR). Young and old MuSCs exhibited similarly high frequencies of expression of the Pax7 and Myf5 stem cell transcripts and infrequent (<15%) expression of Pax3 and MyoD transcripts, markers of committed progenitors, indicating that the two populations were equivalently enriched for MuSCs (FIGS. 1B-C and 6).

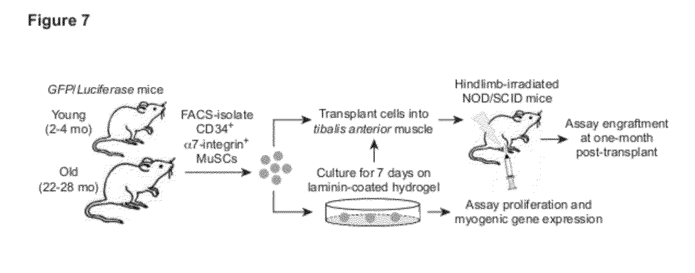

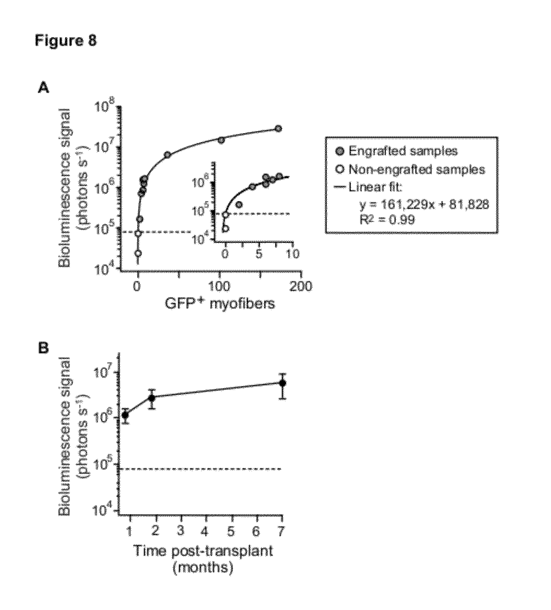

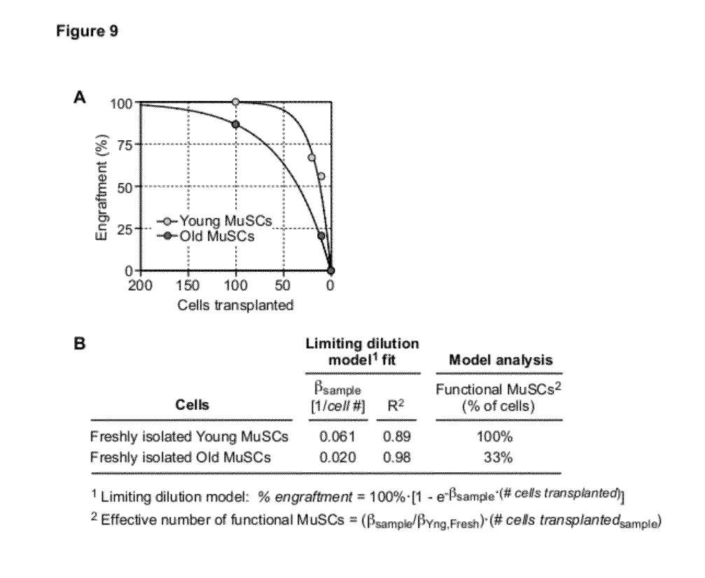

To assay their intrinsic regenerative function, 10 or 100 young or old MuSCs isolated from transgenic GFP/Luciferase mice were injected intramuscularly into irradiated hindlimb muscles of young NOD/SCID mice (FIG. 7). Regeneration was monitored by bioluminescence imaging (BLI) of luciferase activity and histological analysis of GFP expression after four weeks (FIGS. 1D-G and 8A), a time-point that marks the onset of stable long-term engraftment of transplanted MuSCs (FIG. 8B). Upon transplantation of 100 cells, the young and old MuSCs did not differ significantly in their frequency of engraftment (FIG. 1G), in agreement with findings of others. However, when as few as 10 cells were transplanted, a profound difference in the function of young and old MuSCs was observed, revealing that old MuSCs had a diminished regenerative capacity one-third that of young MuSCs (FIG. 1G-H and shown using a classic stem cell limiting dilution model in FIG. 9). These findings were enabled by the study of a very small number of stem cells. The data provide novel evidence that old MuSCs have an inherently impaired capacity to regenerate damaged muscle.

Even after culture on soft hydrogels, a defect in regeneration by old MuSCs was apparent. In our previous studies with young MuSCs maintained on a soft hydrogel with elasticity similar to muscle tissue (12 kPa Young's modulus), the cells retained their stem cell function and self-renewed, whereas on rigid hydrogels with a stiffness similar to plastic (˜10⁶ kPa) these properties were lost. To test their regenerative function in vivo, young and old MuSCs isolated from GFP/Luciferase mice were maintained for one week on rigid or soft hydrogels, and then collected, counted, and 100 cells transplanted into the hindlimbs of recipient mice (FIG. 2A-C). After culture on rigid hydrogels, young MuSCs exhibited minimal engraftment and muscle regeneration, whereas old MuSCs did not engraft. After culture on soft hydrogels, 35% of mice exhibited significant engraftment following transplantation of young MuSCs, compared to only 10% for old MuSCs. Further, a reduced fraction of old MuSCs underwent cell division in clonal assays in soft hydrogel microwells compared to young MuSCs (FIG. 2D-E), similar to reports in other culture systems. These results demonstrate that MuSCs from old mice exhibit a substantial self-renewal defect in culture that is only partially corrected by culture on substrates with elasticity comparable to muscle tissue.

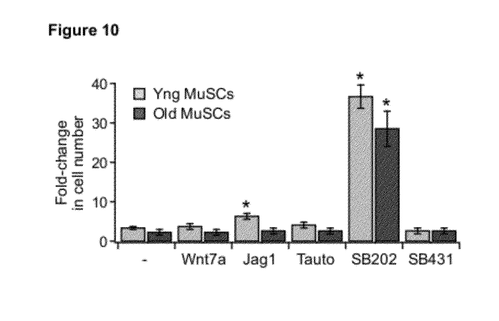

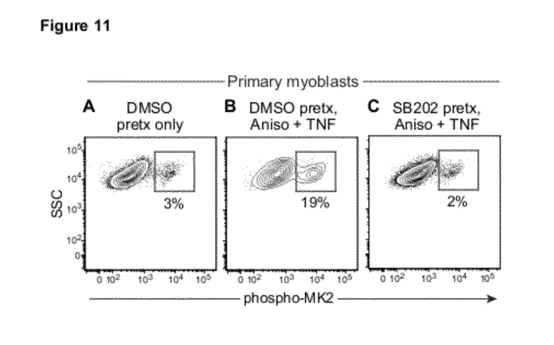

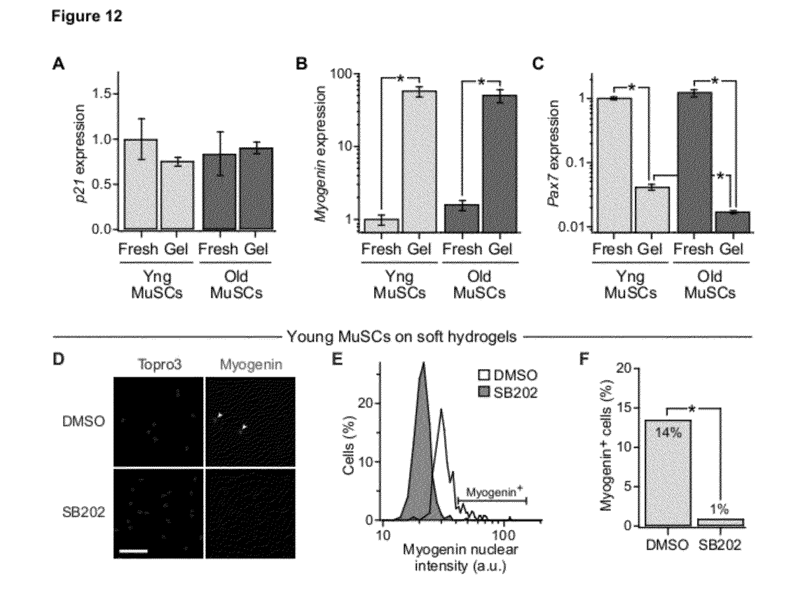

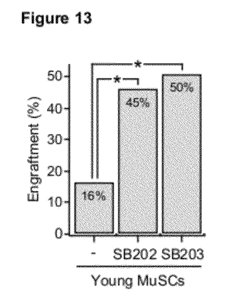

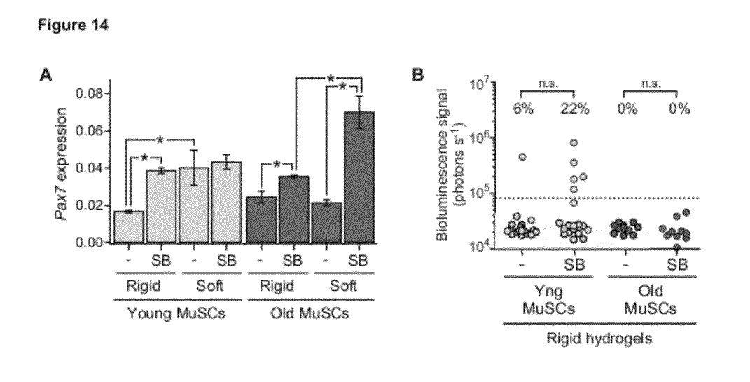

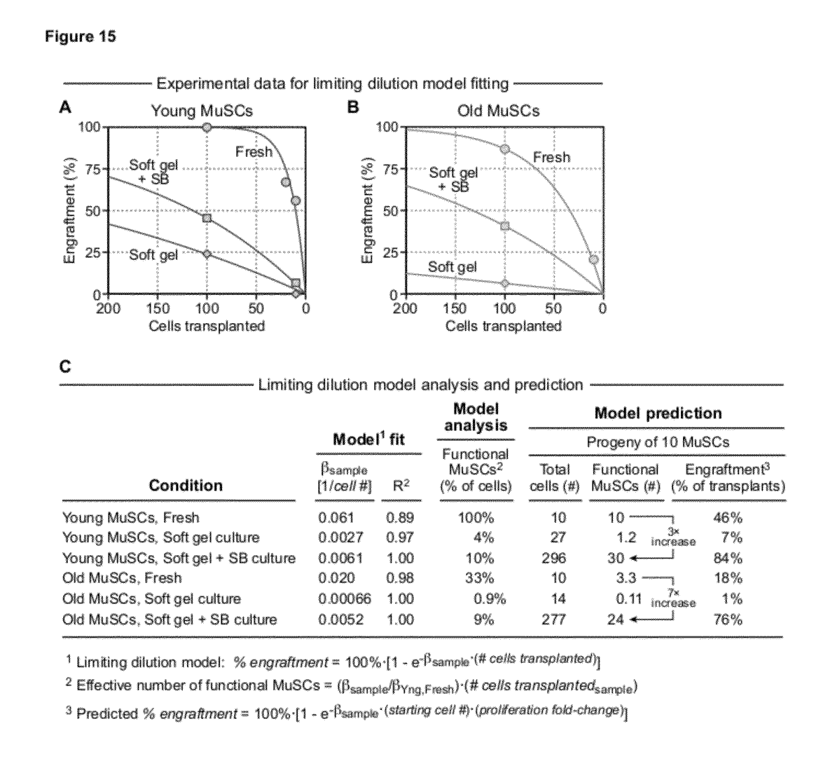

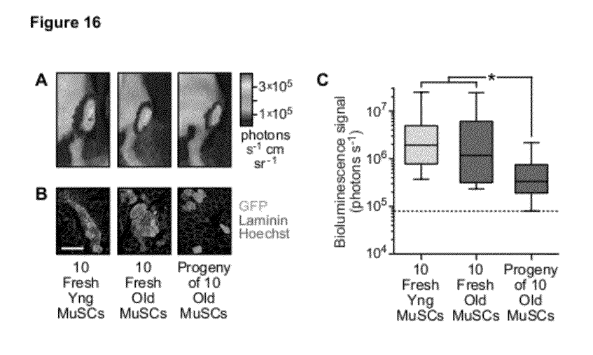

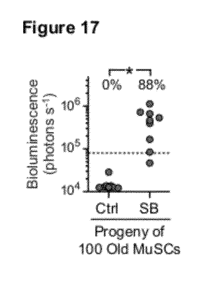

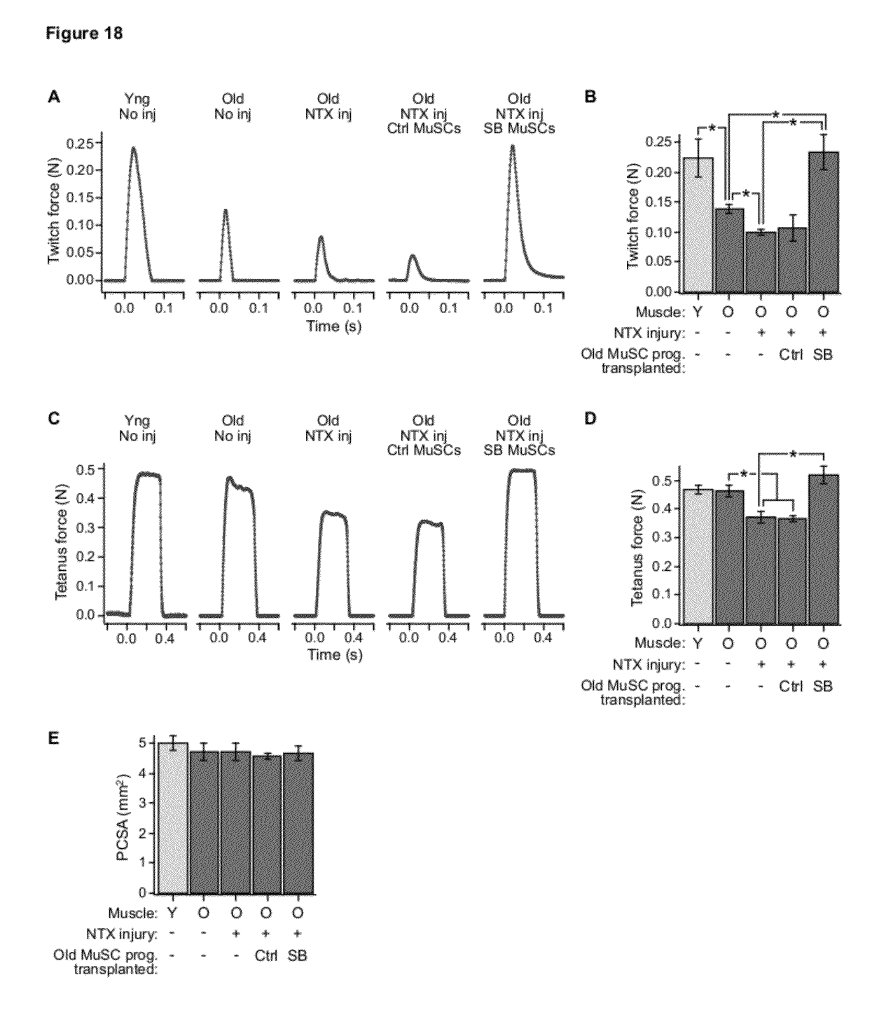

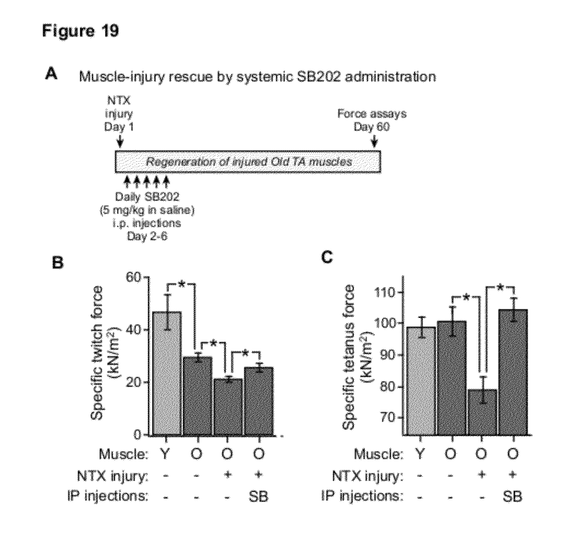

Since a fraction of old MuSCs retained proliferative and regenerative potential on soft hydrogels, we performed an analysis of candidate compounds and proteins that increase proliferation of old MuSCs and augment their regenerative potential. We assayed multiple molecules, including Wnt and Notch activating ligands and chemical inhibitors of MuSC signaling pathways, that have all been previously associated with stimulating young MuSC proliferation and/or self-renewal (Table 1 and FIG. 10). This candidate approach determined that SB202190 (SB), an inhibitor of the α and β isoforms of p38 MAP kinase, stimulated the profound proliferation of both young and old MuSCs (˜30-fold increase in one week relative to seeding; FIG. 2F). Our finding confirms a previous report showing p38 inhibition enhances proliferation of young myogenic progenitors and MuSCs in myofiber explant cultures, and now shows that it is also true for purified old MuSCs. SB substantially attenuated p38 signaling activity in cultured primary mouse myoblasts, as assayed by the phosphorylation of the p38 substrate MK2 (also known as MAPKAPK-2), confirming the pathway specificity of this treatment (FIG. 11).

The data show that p38 inhibition potently enhances stem cell gene expression patterns in young and old MuSCs (FIG. 2G-J). Old MuSCs cultured on soft hydrogels and treated with SB had decreased transcription of the cell cycle inhibitor p21, and the commitment marker Myogenin, and increased transcription of the MuSC transcription factor Pax7 (FIGS. 2G-I and 12). In agreement with previous observations by others with young MuSCs and myoblasts, these data show that p38 inhibition also enhances cell proliferation and induces a stem cell-specific gene expression profile in purified old MuSCs. This data provides previously unreported mechanistic insights implicating p38 signaling as a potential negative regulator of self-renewal in old MuSCs.

To determine if ex vivo SB treatment improves old MuSC function, we tested their ability to regenerate injured muscles in mice. We transplanted 100 cells from cultures of young and old GFP/Luciferase MuSCs after exposure to SB for one week on soft hydrogel (FIG. 3A-C). Remarkably, SB treatment improved the regenerative capacity of both young and old MuSCs to similarly high levels, with an engraftment frequency for 100-cell transplants of 45% and 41%, respectively (FIG. 3C). Similar results were obtained with a different p38 inhibitor, SB203580 (FIG. 13). Notably, rigid hydrogel substrates did not support a similar SB-mediated improvement (FIG. 14). These results demonstrate that SB treatment in combination with a soft hydrogel substrate restores old MuSC function to that of young MuSCs providing the first definitive in vivo functional evidence that old MuSCs can be rejuvenated ex vivo and regenerate muscle on a par with young MuSCs.

To further analyze these data, we developed a mathematical model using a classic limiting dilution stem cell transplantation analysis (FIG. 15). This analysis predicted that SB treatment of old MuSCs results in a 10-fold improvement in the proportion of functional MuSCs, increasing from 0.9% on soft hydrogels alone to 9% on hydrogels with SB treatment (FIG. 15C). This increase in the proportion of functional MuSCs accompanies a substantial (˜30-fold) increase in total cell numbers observed in SB-treated cultures (FIG. 2F). Mathematical modeling incorporating both of these findings showed that the absolute number of functional stem cells increased 7-fold relative to freshly isolated old MuSCs (FIG. 15C), indicative of self-renewal divisions leading to an expansion of the functional stem cell pool.

We tested the mathematical prediction that expansion of functional muscle stem cells had occurred ex vivo. Modeling suggested that the culture progeny of 10 isolated old MuSCs treated with SB should yield engraftment in 76% of transplants, which exceeds that for the same starting number of freshly isolated young or old MuSCs (FIGS. 15C and 3D). Consistent with modeling predictions, following SB treatment, the progeny of 10 old MuSCs yielded a striking 79% transplant engraftment frequency (FIGS. 3E-G). By comparison, transplants of 10 freshly isolated old MuSCs yielded only 33% engraftment, confirming that stem-cell expansion of SB-treated old MuSCs had occurred in culture. Moreover, these data confirmed that ex vivo SB treatment rejuvenated the stem cell population, as the engraftment frequency seen for the culture progeny of SB-treated old MuSCs (79%) was now comparable to that seen with freshly isolated young MuSCs (63%). Although the frequency of engraftment is comparable to young MuSCs, the extent of engraftment, as determined by BLI and histology, is not as robust (FIGS. 3F and 16). Most striking, however, is the novel finding that p38 inhibition in culture leads to rejuvenation, expansion, and increased regenerative function of old MuSCs. Further, old MuSCs expanded ex vivo can home to the native satellite cell niche following transplantation into muscle, an indication that they are capable of replenishing the stem cell pool and responding to successive regenerative demands in transplant recipients (FIG. 3H), an essential characteristic of muscle stem cells.

We tested whether SB-treated old MuSCs are not only capable of regenerating damaged muscle in young mice (FIG. 3), but also in old mice. For this purpose, we developed an autologous cell-therapy model in which old MuSCs from wildtype C57BL/6 mice were cultured on soft hydrogels, infected with a GFP/Luciferase lentivirus, and their cell progeny transplanted into notexin-injured syngeneic old C57BL/6 muscles (FIG. 4A). Our data show that in syngeneic immune-competent old recipients, the transplanted progeny of 100 SB-treated old MuSCs routinely engrafted at a high frequency (88%) in aged recipient muscle, by comparison with 100 non-SB-treated old MuSC progeny (0%; FIGS. 4B-C and 17). This autologous engraftment and rescue of muscles of old mice by SB-treated old MuSCs is also evident histologically by the substantial contribution to myofibers (FIG. 4D).

Finally, we determined whether the contributions of ex vivo SB-treated old MuSCs increased muscle force post-transplant. We measured specific twitch and tetanus forces of notexin-injured muscles from old mice that received syngeneic MuSC transplants at two months post-injury. Force generation was compared to controls, including uninjured muscles from young and old mice and injured muscles from old mice that did not receive MuSC transplantation (FIG. 4E-F). To avoid potential artifacts associated with measurements that employ complete muscle excision, we assayed force generation in intact tibialis anterior muscles in live mice. In uninjured mice, old muscles exhibited reduced twitch forces while tetanus forces were unchanged, in agreement with prior reports that a reduction in fast-twitch (type II) rather than slow-twitch (type I) fibers is observed with aging. As expected, injured old muscles exhibited defective regeneration evident by impaired twitch and tetanus force generation. Remarkably, injured old muscles that received transplants of the progeny of SB-treated old MuSCs exhibited increased force generation that was restored (tetanus) or even exceeded (twitch) pre-injury levels. Similarly, systemic SB treatment by IP injection (5 daily injections starting at one day after injury) aided recovery of both twitch and tetanus forces in injured old mice by two months post-injury (FIG. 19).

In this report, we provide functional evidence that muscle stem cells from aged mice are inherently defective in regeneration relative to MuSCs from young mice. Thus, MuSCs, like other tissue-specific stem cells, exhibit intrinsic aging-associated defects. We show that the intrinsic defects of old stem cells are overcome by transient ex vivo exposure to a p38 inhibitor in combination with culture on soft hydrogel so that they function similarly to young MuSCs in regenerating damaged muscles. Further, the proportion and absolute number of functional stem cells are increased, providing the novel evidence that MuSCs from old mice can be expanded in culture. This treatment overcomes limitations previously seen with cultured myoblasts. Finally, and most striking, is the finding that the strength of old muscles is markedly enhanced following localized transplantation of ex vivo-treated old MuSCs. These data indicate that ex vivo exposure of MuSCs on soft hydrogels to p38 inhibitors, or in vivo exposure to p38 inhibitors can constitute a localized stem cell therapy for muscle atrophy in aged individuals following acute injury.

### Materials & Methods

Animals. All animal protocols were approved by the Stanford University Administrative Panel on Laboratory Animal Care (APLAC) and experiments were performed in compliance with the institutional guidelines of Stanford University. C57BL6 young adult mice were purchased from Jackson Laboratories (Bar Harbor, Me.) and used at 2-4 months of age (median age 2 months). C57BL6 old adult mice were purchased from the National Institute of Aging and used at 22-28 months of age (median 24 months). Mice ubiquitously expressing a green fluorescent protein (GFP) transgene, mice ubiquitously expressing a firefly luciferase (Fluc) transgene driven by the ACTB promoter (L2G85 strain), and mice expressing nLacZ under regulation of the Myf5 promoter (Myf5^{nLacZl+}) were obtained as described previously. Double transgenic GFP/Luciferase and Myf5^{nLacZl+}/Luciferase mice were generated by breeding the above strains and were confirmed by appropriate PCR-based strategies to validate the genotype. Cells from C57BL/6 mice were used for cell proliferation, phosphoprotein, and gene expression experiments. Cells from GFP/Luciferase and Myf5^{nLacZl+}/Luciferase were used for transplantation experiments and were used at 2-4 months of age (median age 2 months) for young mice and 22-28 months of age (median 24 months) for old mice.

Muscle stem cell (MuSC) isolation. Muscle stem cells were isolated as previously described. Briefly, mouse tibialis anterior and gastrocnemius muscles were dissected and subjected to collagenase (0.25%) and dispase (0.04 U/ml; Roche, Indianapolis, Ind.) digestion. Non-muscle tissue was removed under a dissection microscope and muscle fibers were dissociated. After 90 minutes total digestion, the remaining cell suspension was passed through a nylon 70-μm filter (BD Biosciences, San Jose, Calif.). Cells were incubated with biotinylated antibodies reactive to CD45, CD11 b, CD31, and Sca1 (BD Biosciences). Cells were then incubated with streptavidin magnetic beads (Miltenyi Biotech, Auburn, Calif.), streptavidin-Texas Red (Invitrogen, Carlsbad, Calif.), α7-integrin-phycoerithrin antibody (AbLab, Vancouver, Canada), and CD34-eFluor660 antibody (eBioscience, San Diego, Calif.).

After magnetic depletion of the biotin-positive cells, the biotin-negative cells sorted on a modified FACStar Plus cell sorter using FACS Diva software (BD Biosciences). To isolate MuSCs, cells were gated for viable cells (propidium iodide negative) then cells negative for lineage markers CD45, CD31, CD11 b, and Sca1 were gated. Within this population, cells positive for both CD34 and α7-integrin were gated, representing the MuSC fraction. Cells were double-sorted for purity (routinely >95% for C57BL/6 cells). For GFP/Luciferase cells, MuSCs were also gated for GFP positivity and were routinely >90% double-sorted purity. Flow cytometry scatter plots were generated using FlowJo v8.7 (Treestar, Ashland, Oreg.).

Single-cell RT-PCR. Nested multiplexed RT-PCR was performed on freshly sorted single young and old MuSCs as described previously (15), except the following primers were used.

| | Forward Primer (5′→3′) | Reverse Primer (5′→3′) |
|---|---|---|
| Pax7 External | GAGTTCGATTAGCCGAGTGC | GGTTAGCTCCTGCCTGCTTA |
| Myf5 External | AGACGCCTGAAGAAGGTCAA | AGCTGGACACGGAGCTTTTA |
| Pax3 External | AACCATATCCGCCACAAGAT | CTAGATCCGCCTCCTCCTCT |
| MyoD External | TACCCAAGGTGGAGATCCTG | GTGGAGATGCGCTCCACTAT |
| Pax7 Internal | GCGAGAAGAAAGCCAAACAC | GGGTGTAGATGTCCGGGTAG |
| Myf5 Internal | CCACCAACCCTAACCAGAGA | CTGTTCTTTCGGGACCAGAC |
| Pax3 Internal | CCCATGGTTGCGTCTCTAAG | GGATGCGGCTGATAGAACTC |
| MyoD Internal | GCCTTCTACGCACCTGGAC | ACTCTTCCCTGGCCTGGACT |

MuSC transplantation. MuSCs were transplanted immediately following FACS isolation or after collection from cell culture directly into the tibialis anterior muscle of recipient mice as previously described. Cells from GFP/Luciferase or Myf5^{nLacZl+}/Luciferase mice were transplanted into gender-matched, hindlimb-irradiated NOD/SCID (Jackson) mice. NOD/SCID (2-4 months of age, median 2 months) mice were anesthetized with ketamine (2.4 mg per mouse) and xylazine (240 μg per mouse) by intraperitoneal injection then were irradiated by a single dose of 18 Gy administered to the hindlimbs, with the rest of the body shielded in a lead jig. Transplantation was performed within three days post-irradiation. In syngeneic transplant studies, cells from old C57BL/6 mice were transplanted into gender-matched, littermate old C57BL/6 mice (Jackson) following culture for one week on soft hydrogels. Specifically, old C57BL/6 MuSCs were infected with a GFP/Luciferase lentivirus added at day 2 of culture for 24 hours. Recipient old C57BL/6 mice were acutely injured by a single 10 μl intramuscular injection of notexin (10 μg/ml; Latoxan, France) two days prior to cell transplantation, but were not irradiated. Freshly isolated or cultured cells were counted by hemocytometer and resuspended at desired cell concentrations in PBS with 2.5% goat serum and 1 mM EDTA. Cells were collected from hydrogel cultures by incubation with 0.5% trypsin in PBS for 2 min at 37° C. and counted using a hemocytometer. Cells were transplanted by intramuscular injection into the tibialis anterior muscle in a 10 μl volume.

Tissue histology. Recipient tibialis anterior muscle tissues were prepared for histology as previously described to analyze GFP expression. Transverse sections were incubated with anti-laminin (Millipore, Billerica, Calif.), anti-GFP (Invitrogen) antibodies, and/or anti-β-galactosidase (Abcam, Cambridge, Mass.) with appropriate secondary antibodies (Invitrogen). Nuclei were counter-stained with Hoechst 33342 (Invitrogen). Images were acquired with an AxioPlan2 epi-fluorescent microscope (Carl Zeiss Microimaging, Thornwood, N.Y.) with Plan NeoFluar 10×/0.30NA or 20×/0.75NA objectives (Carl Zeiss) and an ORCA-ER digital camera (Hamamatsu Photonics, Bridgewater, N.J.). Digital images were captured in OpenLab software (Improvision, Waltham, Mass.) and assembled in Photoshop software (Adobe, San Jose, Calif.) with consistent contrast adjustments across all images from the same stain.

Bioluminescence imaging. Bioluminescence imaging was performed using a Xenogen-100 system, as previously described. Briefly, mice were anesthetized under isofluorane and 100 μL luciferin (0.1 mmol/kg; Caliper LifeSciences, Hopkinton, Mass.) in PBS was administered by intraperitoneal injection. Following luciferin administration, images were acquired every minute for 15 min. Images acquired at 12 min post-luciferin injection were used for analysis. Digital images were recorded and analyzed using Living Image software (Caliper LifeSciences). Images were analyzed with a consistent region-of-interest (ROI) placed over each hindlimb to calculate a bioluminescence signal. A bioluminescence signal value of 80,000 photons/s was used to define a engraftment threshold, as this level corresponds to the histological detection of one or more GFP⁺ myofiber (see FIG. 8A). BLI imaging was performed one month post-transplant unless otherwise stated.

Limiting dilution analysis and model prediction. To calculate the effective number of functional MuSCs in any transplanted cell population, the transplant engraftment efficiency was related to the number of cells transplanted (across a range of transplanted cell numbers) using an exponential stem-cell limiting dilution model:

% engraftment=100%·[1-exp [(βₛₐₘₚₗₑ)·(# cells transplantedₛₐₘₚₗₑ)] [eqn. 1]

where β is an exponential coefficient fit from experimental data. This model fit well to experimental data from a variety of cell conditions (R²=0.89-1.00; see FIG. 16C). Using this model, the number of functional MuSCs (e.g. defined as equivalent to freshly isolated young MuSCs) within any given population can be calculated:

# Functional MuSCsₛₐₘₚₗₑ=(β_{Sample}/β_{Yng,Fresh})˜(# cellsₛₐₘₚₗₑ). [eqn. 2]

The number of functional MuSCs arising from the progeny of each MuSC initially seeded in the cell culture experiments can be predicted by taking into account the measured cell proliferation in culture (see FIG. 2D). The transplant engraftment percentage of the cell culture progeny from any starting MuSC cell number can be predicted using the measured cell proliferation data and the fit exponential model parameter β:

Predicted % engraftment=100%·[1-exp [(βₛₐₘₚₗₑ)·(starting cell #)·(proliferation fold-changeₛₐₘₚₗₑ)]]. [Eqn. 3]

Hydrogel fabrication. Poly(ethylene glycol) (PEG) hydrogels were fabricated from precursors synthesized as described previously. Briefly, hydrogels were produced by mixing PEG-sulfhydryl (4-arm 10 kDa PEG-SH) and PEG-vinylsulfone (8-arm 10 kDa PEG-VS) precursors in water or triethanolamine, respectively. Hydrogel surfaces were tethered through covalent cross-linking reaction with PBS-dialyzed laminin protein (Roche). Soft 12 kPa (Young's modulus) stiffness hydrogels were fabricated to 1 mm thickness. Rigid ˜10⁶ kPa stiffness hydrogels were fabricated to a ˜1-2 μm thickness directly onto tissue-culture plastic dishes. The non-swelling gel chemistry results in equivalent laminin protein coating concentrations (˜7.5 ng/cm²) on both soft and rigid hydrogels. Hydrogels of ≦1 μm thickness allow cells to sense the underlying substrate mechanical properties, resulting in an effective rigidity equivalent to tissue-culture plastic. Hydrogel microwell arrays for clonal proliferation experiments were fabricated as described previously. All hydrogels were cut and glued to fill the surfaces (2.0 cm² area) of 24-well culture plate wells. See, for example, co-pending application WO 2012/009682, herein specifically incorporated by reference.

MuSC culture and treatment. Following isolation, MuSCs were pelleted and resuspended into myogenic cell culture medium containing DMEM/F10 (50:50), 15% FBS, 2.5 ng/ml bFGF, and 1% penicillin/streptomycin. MuSCs were seeded at 1000 cells per well (24-well plate size wells, 2.0 cm² surface area) on laminin-tethered hydrogels of either 12 kPa (soft) or ˜10⁶ kPa (rigid) rigidities. Cells were maintained at 37° C. in 5% CO₂. Media was changed daily. For proliferation and p38 inhibition studies, the following ligands/inhibitors were supplemented in the myogenic medium and maintained consistently through the experiment: 25 ng/ml Wnt7a (R&D Systems, Minneapolis, Minn.), 25 ng/ml Jagged1-Fc (R&D Systems, Minneapolis, Minn.), 10 nM tautomycetin (PP1 inhibitor; Tocris Bioscience, Ellisville, Mo.), 10 μM SB202190 or 10 μM SB203580 (p38 inhibitors; EMD Chemicals, Gibbstown, N.J.), 1 μM SB431542 (ALK4/5/7 inhibitor; Tocris Bioscience), or 0.1% DMSO carrier control. All MuSC cell culture assays and transplantations were performed after one week of culture unless noted otherwise.

Proliferation assays. MuSC proliferation in hydrogel culture was assessed at both the clonal and bulk levels. To assay clonal proliferation, MuSCs were seeded at 1000 cells per 2.0 cm² hydrogel microwell arrays with 150 μm diameter microwells. This seeding density resulted in >90% attached cells to adhere in a microwell independent of other cells. Cell proliferation was monitored by time-lapse microscopy from 12 hours (day 0) to four days post-seeding as described previously using a PALM/AxioObserver Z1 system (Carl Zeiss MicroImaging) with custom environmental control chamber and motorized stage. Images were recorded every 20 min at 5× magnification. Image sequences were processed to find viable, motile cells (>90% of all adherent cells for both young and old MuSCs) using the custom Baxter image-analysis toolbox in Matlab (Mathworks, Natick, Mass.). After identification of microwells with viable clones, sequences were manually viewed using the Baxter toolbox to score clones with evident cell division by day 4 of culture. To assay bulk proliferation, MuSCs were seeded at 1000 cells per 2.0 cm² flat hydrogels. After one week, cells were trypsinized and counted on a hemocytometer.

Phospho-MK2 flow cytometry. To analyze p38 signaling, the phosphorylation of MK2, a readout of p38 kinase activity, was assayed by flow cytometry. Primary mouse myoblasts were isolated as previously described. Myoblasts were cultured on collagen-coated tissue-culture plastic dishes in myogenic cell medium and were maintained at 37° C. in 5% CO₂. Myoblasts were pretreated with 0.1% DMSO carrier control or 10 μM SB202190 for one-hour and then were stimulated with anisomycin (1 μg/ml; Sigma) and TNF (50 ng/ml; R&D Systems, Minneapolis, Minn.) for 30 minutes or mock buffer (PBS). Pretreatments and stimuli were added in 50× spike solutions. After 15 minutes, cells were collected from hydrogel cultures by incubation 0.5% trypsin in PBS for 2 min at 37° C. Cells were fixed with 1.5% paraformaldehyde in PBS, permeabilized in methanol at −80° C., and stained with an AlexFluor488-conjugated antibody reactive to the phosphorylated (Thr334) form of MK2 (Cell Signaling). Cells were analyzed for phospho-MK2 positivity on a modified FACStar Plus flow cytometer using FACS Diva software (BD Biosciences).

Quantitative real-time PCR. RNA was isolated from freshly isolated and cultured cells using the RNeasy Micro kit (Qiagen, Valencia, Calif.). Total RNA from each sample was reverse transcribed using the High Capacity cDNA RT kit (Applied Biosystems, Carlsbad, Calif.). cDNA was subjected to real-time PCR using a SYBR Green PCR Master Mix (Applied Biosystems). Real-time PCR was performed using an ABI 7900HT Real Time PCR system and software (Applied Biosystems). Samples were cycled at 95° C. for 10 min and then 40 cycles of 95° C. for 15 sec and 60° C. for 1 min. To quantify relative transcript levels, the 2^{-ΔΔCt} method was used, and freshly isolated young MuSCs were used as reference value (plotted as 1 in all graphs). Gapdh was used as a normalizing gene. Primer sequences for Gapdh, p21, myogenin, and Pax7 were obtained from literature or using NIH Primer3.

| Gene | Forward Primer (5′→3′) | Reverse Primer (5′→3′) |
|---|---|---|
| Gapdh | CACTGAGCATCTCCCTCACA | TGGGTGCAGCGAACTTTATT |
| p21 | AGCCTGAAGACTGTGATGGG | AAAGTTCCACCGTTCTCGG |
| Myogenin | TGTTTGTAAAGCTGCCGTCTGA | CCTGCCTGTTCCCGGTATC |
| Pax7 | CTGGATGAGGGCTCAGATGT | GGTTAGCTCCTGCCTGCTTA |

Myogenin immunofluorescence analysis. MuSCs from young mice were isolated and cultured on 12 kPa hydrogels with either 0.1% DMSO control or 10 μM SB202190. After one week of culture, cells were fixed with 0.5% paraformaldehyde in PBS. Fixed cells were blocked with 20% goat serum and 0.5% Triton X100 in PBS. Cells were stained with anti-myogenin antibody (Santa Cruz Biotechnology, Santa Cruz, Calif.) and then an AlexaFluor594-conjugated secondary antibody (Invitrogen). Nuclei were stained with Topro3 (Invitrogen)the Immunofluorescence images were acquired using a LSM510 laser-scanning confocal microscope (Carl Zeiss Microimaging) with a Plan NeoFluar 20x/0.75NA objective. Digital images were captured using LSM510 software. Images were composed and edited in Photoshop software (Adobe). Background was reduced and contrast was enhanced equivalently across all images of the same stain. Nuclear regions were identified by Topro3 staining using MetaMorph software (Molecular Devices, Sunnyvale, Calif.). Within each nuclear region, the myogenin intensity was integrated using MetaMorph software. Nuclear myogenin intensities from each condition were plotted in a log-scale histogram to establish a threshold defining myogenin-positive cells.

Lentiviral infection. Lentivirus infection was carried on MuSCs cultured (1000 cells seeded in a 2 cm² culture well) on soft hydrogels from 24 to 48 hours post-seeding. A EF1α promoter-driven Luciferase-IRES-GFP lentivirus was generated as described previously. For infection, the GFP/Luciferase lentivirus was diluted to 10,000 U/ml (10 MOI) in myogenic cell culture medium (1 ml/well) supplemented with 4 μg/ml protamine sulfate.

Systemic SB202190 administration. Old mice were systemically administered SB202190 (5 mg/kg body weight in 0.9% NaCl/H₂O) with five daily doses by IP injection starting at one day after NTX injury.

In vivo force measurements. Force measurements were performed on tibialis anterior (TA) muscles in vivo in anesthetized mice, as previously described. The hindlimb was shaved and fixed to a frame to immobilize it without compromising the blood supply to the leg. The animal was warmed on an isothermal pad and by heat lamp. A small incision was placed in the skin directly above the TA muscle. The distal tendon was sutured to a thin metal hook and then attached to a force transducer (300C-LR; Aurora Scientific, Aurora, Ontario). Distal tendons from all front lower hindlimb muscles other than TA were cut, leaving TA isolated during attachment to the force transducer. The muscles and tendons were kept moist by periodic wetting with saline (0.9% sodium chloride) solution. Then a bipolar electrical stimulation cuff was placed around the central third of the TA. In all measurements, 0.1 ms pulses at predetermined supra-maximal stimulation intensity were used. During twitch force measurements, the muscle was stimulated with a single 0.1 ms pulse. During tetanic force measurements, the muscle was stimulated at 150 Hz for 0.3 s. Muscle force and synchronization pulses were recorded for 2 seconds immediately prior to the stimulation and 3 seconds after the end of the stimulation. Five twitch and then five tetanic measurements were performed on each muscle, with 3-5 minutes between each measurement. Data were collected with a PCI-6251 data acquisition card (National Instruments, Austin, Tex.) and analyzed in Matlab. Specific force values were calculated by normalizing the force measurements by the muscle physiological cross-sectional area (PCSA), as described previously:

PCSA=(muscle volume/fiber length)×(cos φ_{fibers}). [eqn. 4]

where φ_{fibers} is the pennation angle of the muscle fibers. Muscle volume was determined by displacement and fiber length was measured with micrometer.

Statistical analysis. Cell culture experiments were performed with at least three replicates. For single-cell gene expression, proliferation, and immunofluorescence assays, the number of individual cells quantified are reported in figure legends. Transplantation experiments were performed in at least three independent experiments, unless otherwise noted, with at least 10 total transplants per condition. For single-cell assays and transplant engraftment data, a Fisher's exact test was used for comparisons between conditions. For comparing BLI signal levels for engrafted transplants, an unpaired, two-tailed Mann-Whitney test was used due to non-normality in values. For bulk proliferation, gene expression, and force assays, an unpaired, two-tailed t test was used. A significance level of α=0.05 was used for all tests.

What is claimed is:

1. A method for improving the function of aged, diseased, and/or injured tissues in an individual, the method comprising:
contacting somatic stem cells with an inhibitor of the p38 MAPK signaling pathway;
wherein said stem cells are induced and/or transplanted to regenerate said tissue.

2. The method according to claim 1, wherein said stem cells are resident in said tissue.

3. The method according to claim 2, wherein said stem cells are induced to proliferate, wherein the progeny of said stem cells differentiate and regenerate said tissue.

4. The method according to claim 1, wherein said tissue regenerating agent is administered locally or systemically to the individual.

5. The method of claim 4, wherein the tissue regenerating agent is administered by direct injection systemically.

6. The method of claim 4, wherein the tissue regenerating agent is administered by direct injection intramuscularly.

7. The method of claim 1, wherein the source of said somatic stem cells is selected from bone marrow, blood, the cornea and the retina of the eye, brain, skeletal muscle, cartilage, bones, dental pulp, liver, skin, the lining of the gastrointestinal tract, and pancreas.

8. The method according to claim 7, wherein said stem cell is a muscle satellite cell.

9. The method according to claim 7, wherein said stem cell is a hematopoietic stem cell.

10. The method according to claim 7, wherein said stem cell is a neural stem cell.

11. The method according to claim 7, wherein said stem cell is a mesenchymal stem cell.

12. The method according to claim 1, wherein said p38 pathway inhibitor inhibits p38 MAPK.

13. The method according to claim 12, wherein said inhibitor inhibits p38 MAPK alpha and/or beta.

14. The method of claim 12, wherein said inhibitor is a small molecule inhibitor.

15. The method of claim 12, wherein said inhibitor is a polynucleotide.

16. The method of claim 1 wherein said somatic stem cell is present in an ex vivo culture.

17. The method of claim 16, wherein said ex vivo culture comprises a pliable substrate.

18. The method of claim 17, wherein said pliable substrate is a hydrogel.

19. The method according to claim 16, wherein said stem cells are expanded in culture.

20. The method of claim 19, wherein said expanded stem cells are transplanted in to a recipient.

21. The method according to claim 16, wherein said stem cell is a muscle satellite cell.

22. The method according to claim 16, wherein said stem cell is a hematopoietic stem cell.

23. The method according to claim 16, wherein said stem cell is a neural stem cell.

24. The method according to claim 16, wherein said stem cell is a mesenchymal stem cell.

25. The method according to claim 16, wherein said p38 pathway inhibitor inhibits p38 MAPK.

26. The method according to claim 25, wherein said inhibitor inhibits p38 MAPK alpha and/or beta.

27. The method of claim 25, wherein said inhibitor is a small molecule inhibitor.

28. The method of claim 25, wherein said inhibitor is a polynucleotide.

29. The method of claim 1, wherein said inhibitor acts on upstream or downstream elements of the p38 MAPK pathway.

30. The method of claim 11, wherein wherein said inhibitor acts on upstream or downstream elements of the p38 MAPK pathway.
